(19) United States
(12) Patent Application Publication
(10) Pub. No.: US 2021/0317189 A1
(43) Pub. Date: Oct. 14, 2021

(54) FORMULATIONS OF IMMUNOGLOBULIN A

(71) Applicant: Ventria Bioscience Inc., Junction City, KS (US)

(72) Inventors: Solomon Okbazghi, Junction City, KS (US); Vladimir Akoyev, Junction City, KS (US); Saurav Misra, Junction City, KS (US); Randall Alfano, Junction City, KS (US)

(21) Appl. No.: 17/273,784

(22) PCT Filed: Sep. 5, 2019
(86) PCT No.: PCT/US2019/049709

§ 371 Date: Mar. 5, 2021

Related U.S. Application Data
(60) Provisional application No. 62/780,544, filed on Dec. 17, 2018.
(60) Provisional application No. 62/727,345, filed on Sep. 5, 2018.

(51) Int. Cl. C07K 16/00; A23L 33/18; A61K 47/26

(52) U.S. Cl. CPC C07K 16/00; A61K 2039/545; A61K 47/26; A23L 33/18

(57) ABSTRACT

Stabilized formulations for Immunoglobulin A and other biotherapeutic proteins. Formulations comprising at least one pH buffering agent at about pH 5 to about 8, optional non-ionic surfactant, and one or more optional stabilizing agents selected from the group consisting of amino acids, sugars/polyols, chloride salts, carboxylic acids, detergents, natural proteins, protein expression extracts, and mixtures thereof.

### CROSS-REFERENCE TO RELATED APPLICATIONS

The present application claims the priority benefit of U.S. Provisional Patent Application Ser. No. 62/727,345, filed Sep. 5, 2018, entitled STABLE FORMULATIONS OF IMMUNOGLOBULIN A, and U.S. Provisional Patent Application Ser. No. 62/780,544, filed Dec. 17, 2018, entitled ORAL FORMULATIONS OF IMMUNOGLOBULIN A, each of which is incorporated by reference in its entirety herein.

### SEQUENCE LISTING

The following application contains a sequence listing in computer readable format (CRF), submitted as a text file in ASCII format entitled “SequenceListing,” created on Sep. 4, 2019, as 22 KB. The content of the CRF is hereby incorporated by reference.

### BACKGROUND OF THE INVENTION

### Field of the Invention

The present invention focuses on the novel formulation of immunoglobulin A (IgA), including secretory immunoglobulin A (sIgA), and other therapeutic proteins with improved storage stability and/or oral stability.

### Description of Related Art

Protein molecules undergo chemical degradation under various stress conditions, such as harsh acidic and basic pH conditions encountered during elution and neutralization. In addition, the specific type of excipients and/or salts present in the buffer solution may influence the chemical degradation of a protein. Common chemical modifications and causes of degradation of protein molecules include deamidation, racemization, hydrolysis, oxidation, beta-elimination, and disulfide-exchange (i.e., disulfide scrambling), as well as proteolysis. Aside from pH, elevated temperature during storage, shipping, and handling can accelerate chemical degradation. Collectively, our knowledge base regarding chemical modification has increased significantly over the last decade. Several studies have shown that deamidation is a major route of degradation that involves the hydrolysis of Asn and Gln side chain amides and, consequently, leads to changes in charge profile and fragmentation. Such chemical modifications cause loss of functional activity, trigger immunogenicity, and lead to physical instability.

Protein molecules are generally sensitive to stress conditions, such as thermal stress, shear stresses, water-air interfacial stresses, and pH-induced stresses (i.e., stresses induced by suboptimal acidic/basic environments), all of which can lead to aggregation, denaturation, and precipitation. Physical instability is often associated with soluble or insoluble aggregation of the protein molecules, which may induce immunogenicity, generation of anti-drug antibodies, and loss of activity and/or therapeutic potency. For instance, protein molecules are exposed to physical stress during manufacturing processes such as filtration/diafiltration (UF/DF), and purification (e.g., affinity chromatography), which often involves high/low pH shifts during elution and neutralization. Besides manufacturing processes, exposure of a protein molecule to elevated temperature during shipping, storage, or administration could result in physical aggregation and may also ultimately lead to loss of potency. Therefore, there is a need to have a stable liquid formulation at all stages of biologic therapeutic development including pre-clinical and clinical studies.

For therapeutic efficacy, protein-based drugs and biologics must maintain their structural and functional integrity not just during manufacturing and storage but also upon administration and systemic or local delivery within the body. Stability during delivery is critical, for example, to treat diseases of the digestive tract including inflammatory bowel diseases, auto-immune disorders, irritable bowel syndrome, and infectious diseases, all of which cause significant morbidity and mortality. There is a significant need for localized and effective digestive disease treatment using drug products with minimal side effects. Protein molecules encounter physical, chemical and proteolytic degradation during delivery to a targeted site in the GI tract. The harsh acidic pH of the stomach coupled with proteolytic degradation by high concentrations of pepsin creates a formidable initial challenge for oral delivery of biologic therapeutics, such as proteins. This stage is followed by transit from the stomach to the small intestine, which is near basic pH and contains proteolytic enzymes such as trypsin, chymotrypsin, and amylase among others that degrade proteins into smaller peptides. Similarly, after transit from the small to the large intestine, bacterially-derived proteases degrade orally delivered proteins.

Immunoglobulin A (IgA) antibodies play essential roles in the immune function of mucous membranes and protect against pathogenic microorganism and antigens by limiting the access to mucosal barriers. In some cases, IgA directly interacts with pathogenic microorganisms to neutralize their pathogenic capacity. IgA also plays a role in allowing beneficial microbes to colonize the gastrointestinal tract, which has been shown to lead to improved health outcomes in mammals.

IgA comprises four polypeptide chains: two alpha (α) heavy chains and two kappa (κ) light chains (SEQ ID NO:1-2), interconnected by disulfide bonds. Representative sequences are disclosed herein. Immunoglobulin A (IgA) includes the IgA1 (SEQ ID NO:3-4, variable and constant) and IgA2 (IgA2 m1 (SEQ ID NO:5), IgA2m2 (SEQ ID NO:6), and IgA2n (SEQ ID NO:7), allotypes, distinguished by differing a heavy chains) subclasses produced from different sources. Immunoglobulin A could represent a single component or a mixture of monomeric, dimeric, and secretory IgA. The J chain (SEQ ID NO:8) and a secretory component (SC) (SEQ ID NO:9) form intermolecular disulfides that link monomeric IgA into larger assemblies termed dimeric and secretory IgAs. The dimeric forms of IgA comprise two IgA monomers joined together by a single J-chain, but lack the secretory component. Dimeric IgAs may further bind single copies of the secretory component to form sIgA1 and sIgA2 (including sIgA2 m1, sIgA2m2 and IgA2n allotypes). Based on biosynthetic, environmental and stress conditions, as described above, the IgA molecule could contain different post-translational modifications such as glycosylated and non-glycosylated variants, deamidated, oxidized, phosphorylated, glycated and other chemical modifications and minor mutations.

The contribution of IgA in the maintenance of homeostasis is mediated through immunological exclusion, anti-inflammatory properties, and homeostasis of commensals. This is highlighted by the fact that IgA is present at high levels in maternal milk to prevent neonatal bacterial infection. IgA in human milk is estimated to be composed of 70% IgA1 and 30% IgA2 subclasses. Types of sIgA that are highly enriched on the mucosal surfaces of the human body are of particular significance due to their likely robustness in the mucosal environment, potentially high therapeutic efficacies, potentially favorable pharmacological profiles and ability to activate immune response pathways inaccessible to other immunoglobulins. As many pathogenic infectious agents and diseases engage the human body at the mucosa, the development of sIgAs as therapeutic agents that can be delivered at suitable doses is an area of great interest. Such efforts, however, are severely hampered by the lack of stable IgA formulations.

In general, oral delivery of therapeutics is more convenient than infusion or injection, and provides enhanced safety by targeting delivery of a therapeutic directly to the gastrointestinal (GI) tract. However, stability of a therapeutic amount of IgA in gastric and intestinal conditions has been challenging due to the harsh acidic and proteolytic environment of the gastrointestinal tract. The need for stable oral formulations that can deliver IgA to the gastrointestinal tract for treatment of disease would be a significant for advancement of therapies for infectious disease, auto-immune disease, inflammatory disease, metabolic syndrome, obesity, microbiome-mediated diseases, and other conditions. Moreover, therapeutic IgAs can play a significant role to establish or re-establish healthy gastrointestinal microbiomes as primary or supportive treatments for such conditions.

Formulations that protect IgA from chemical and enzymatic degradation and are thus critically required to effectively deliver a therapeutic dose for treatment of various diseases in the intestine and colon. Oral delivery of IgA is beneficial because it provides localized delivery and treatment of infection and inflammation, without systemic immune suppression, and with minimal potential side effects. Furthermore, due to its large molecular weight (380 kDa) systemic absorption of the orally delivered IgA into the bloodstream is less likely, as it is sequestered in the lumenal compartments or surfaces of the GI tract. Thus, a better safety profile compared to current standard therapeutic delivery methods such as infusion, subcutaneous injection or related delivery modes is anticipated.

### SUMMARY OF THE INVENTION

The formulations described herein preserve the native structure/conformation and prevent aggregation, fragmentation, and precipitation of IgA during storage. The described formulations confer stability against thermal stress, freeze-thaw, and/or agitation. Moreover, certain formulations described herein are specifically designed to prevent acidic and proteolytic degradation of IgA, such as in the GI tract. The present invention details formulations of IgA composed of pH buffering agents in combination with one or more additional stabilizing agents, which purposefully improve IgA storage and handling stability and further improve stability in the gastrointestinal tract. Improving the stability is expected to improve patient convenience, by decreasing the frequency of administration and reducing the therapeutic dose required for treatment, and consequently improve patient compliance.

Formulations developed here improve the physical, chemical, and proteolytic stability of IgA, while maintaining its functional activity. Overall, IgA described here is used for treatment of infectious and inflammatory diseases that are known to involve significant damage to the GI tract. Such diseases include ulcerative colitis, celiac disease, and Crohn's diseases (inflammatory bowel diseases), bacterial and viral infectious diseases, and related conditions that are known to cause substantial health problems. Further, oral delivery of IgA targeted toward healthy establishment or re-establishment of the gut microbiome would be a novel application of the technology.

In one aspect, described herein are stabilized prophylactic and/or therapeutic formulations comprising IgA dispersed in a pH buffering agent, at a pH of from about 5 to about 8, wherein the formulation further comprises a non-ionic surfactant and one or more additional stabilizing agents, such that the formulation exhibits physical and chemical stability after mechanical agitation and/or a freeze/thaw cycle. Preferably, the formulation further exhibits oral stability.

In one aspect, methods of using such formulations for prophylactic and/or therapeutic treatments methods are also described herein. Methods include oral delivery of neutralizing immunoglobulins. In one or more embodiments, such immunoglobulins bind and neutralize infectious agents (and/or their virulence factors, surface antigens, or host attachment factors), pro-inflammatory cytokines or their receptors, growth factors/mitogenic factors or their receptors, integrins, cellular attachment and junctional proteins, tumor antigens, biomarkers and proteins, and the like to inhibit and/or reduce the symptoms or severity of a variety of conditions and diseases. Further, emerging research suggests that a number of conditions propagate from the GI tract along the parasympathetic and sympathetic pathways to other areas of the body, such as the brainstem, resulting in a variety of conditions previously thought unrelated to the GI tract. For example, alpha-synuclein aggregation to form insoluble fibrils is a hallmark of a number of synucleinopathies, such as such as Parkinson's disease, dementia with Lewy bodies and multiple system atrophy. However, GI symptoms of such inclusions can be detected decades before diagnosis of the disease. Thus, orally stable formulations described herein have promising use as disease modifying therapies targeting early pathology of such conditions in the gastrointestinal tract, such as by inhibiting alpha-synuclein aggregation.

### BRIEF DESCRIPTION OF THE FIGURES

FIG. 1. Ven-A recovery under tested pH and buffer conditions after incubation for 10 days at 40° C. was measured by denaturing SEC. Control (100%) was the fresh unstressed stock sample of the Ven-A in the Citrate-Phosphate buffer pH 3.7 which was used for preparing samples tested under stress conditions. Recovered Ven-A was expressed as the percent of the total area under the SEC chromatogram.

FIG. 2. Ven-A recovery under tested pH and buffer conditions after incubation for 10 days at 40° C., 60% RH was measured by native SEC. Control (100%) was from unstressed stock sample of the Ven-A in the Citrate-Phosphate buffer, pH 3.7 which was used for preparing samples tested under stress conditions. Recovered Ven-A was expressed as the percent of the total area under the SEC chromatogram.

FIG. 3. Ven-A degree of modifications under tested pH and buffer conditions after incubation for 10 days at 40° C., 60% RH was measured by CEX. Recovered Ven-A was expressed as the percent of the total area under the CEX chromatogram.

FIG. 4. The effect of pH on Ven-A thermostability (on Ven-A denaturation temperature, Tm) after incubation for 10 days at 40° C., 60% RH was measured by Differential Scanning Microcalorimetry (DSC).

FIG. 5. The effect of pH on Ven-A potency in the L929 cell-based assay with hTNF-α after incubation for 10 days at 40° C. Positive control—Adalimumab IgG, Isotype control—sIgA, Control—Ven-A Reference Standard.

FIG. 6. The effect of pH on Ven-A potency in the L929 cell-based assay with mTNF-α after incubation for 10 days at 40° C. Positive control—Adalimumab IgG, Isotype control—sIgA, Control—Ven-A Reference Standard.

FIG. 7. Ven-A recovery under different buffer conditions after incubation for 10 days at 40° C. was measured by SEC. Recovered Ven-A was expressed as the percent of the total area under the SEC chromatogram.

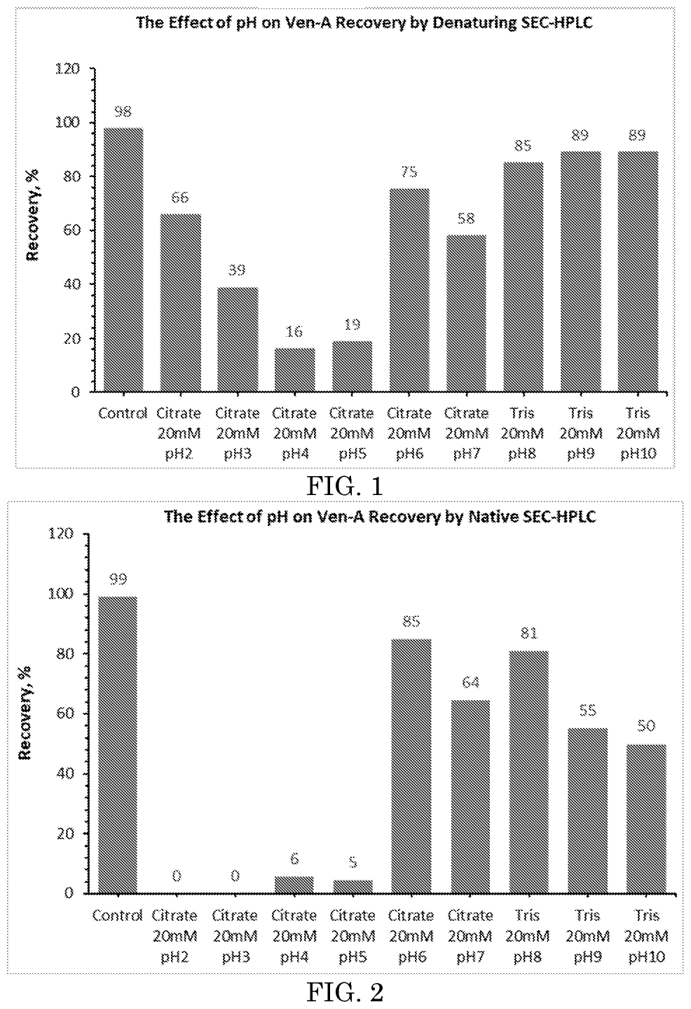

FIG. 1. The effect of buffers (pH 6) on Ven-A Temperature of Denaturation (Tm ° C.) by DSC. Ven-A was incubated under different buffer conditions for 10 days at 40° C.

FIG. 9. The effect of stabilizing agents on Ven-A recovery in the Potassium-Phosphate buffer at pH 6.0 after incubation for 10 days at 40° C. was measured by SEC. Recovered Ven-A was expressed as the percent of the total area under the SEC chromatogram. Precipitation (ppt) was observed in formulation containing tartrate during sample preparation and no further analysis was conducted.

FIG. 10. The effect of stabilizing agents on Ven-A HMWS aggregation and LMWS fragmentation in the Potassium-Phosphate pH 6.0 buffer after incubation for 10 days at 40° C. was measured by SEC. Precipitation (ppt) was observed in formulation containing tartrate during sample preparation and no further analysis was conducted.

FIG. 11. The effect of stabilizing agents on Ven-A recovery in the Potassium-Phosphate pH 6.0 buffer after incubation for 10 days at 40° C. was measured by UV-VIS. Precipitation (ppt) was observed in formulation containing tartrate during sample preparation and no further analysis was conducted.

FIG. 12. The effect of stabilizing agents on Ven-A recovery in the Potassium-Phosphate pH 6.0 buffer after incubation for 10 days at 40° C. was measured by SEC. Recovered Ven-A was expressed as the percent of the total area under the SEC chromatogram.

FIG. 13. The effect of stabilizing agents on Ven-A recovery in the Potassium-Phosphate pH 6.0 buffer after incubation for 10 days at 40° C. was measured by UV-VIS.

FIG. 14. The effect of stabilizing agents on Ven-A recovery (%) after freeze-thaw was measured by SEC.

FIG. 15. The effect of stabilizing agents on Ven-A recovery (%) after agitation was measured by SEC.

FIG. 16. The effect of stabilizing agents on Ven-A recovery after freeze-thaw was measured by UV-VIS.

FIG. 17. The effect of stabilizing agents on Ven-A recovery after agitation was measured by UV-VIS.

FIG. 18. The effect of Polysorbate-80 on Ven-A recovery. The effect of Polysorbate-80 on Ven-A recovery (%) was measured by UV-VIS.

FIG. 19. The effect of Polysorbate-80 on Ven-A recovery (%) was measured by SEC.

FIG. 20. Ven-A recovery (%) in the selected formulations was measured by UV-VIS.

FIG. 21. Ven-A potency in the selected formulations was measured by L929 cells-based assay with mTNF-α.

FIG. 22. Ven-A potency in the selected formulations was measured by L929 cells-based assay with hTNF-α.

FIG. 23. Recovery of high concentration formulations of Ven-A in selected formulations after freeze-thaw and agitation was measured by UV-VIS.

FIG. 24. Stability of IgA formulations (Table 5) in SIF. Stabilizing agent proteins were added to the formulations to stabilize IgA.

FIG. 25. Stability of IgA formulation (Table 5) in SGF. Stabilizing agent proteins were added to the formulations to stabilize IgA.

FIG. 26. Stability of IgA formulations (Table 6) in SGF. Surfactants were added to the formulations to stabilize IgA.

FIG. 27. Stability of IgA formulations (Table 6) in SIF. Surfactants were added to the formulations to stabilize IgA.

FIG. 28. Stability of IgA formulations (Table 7) in SGF. Buffers at different strengths and pHs were tested to prevent proteolytic degradation of IgA.

FIG. 29. Stability of IgA formulations (Table 8) in simulated gastric (SGF) and intestinal fluids (SIF). Expression system extracts with unprocessed IgA were used to prevent proteolytic degradation of IgA formulations.

FIG. 30. Clinical effectiveness of in vivo model. Stabilized sIgA (Ven-B) formulation administered orally as compared to control formulations.

### DETAILED DESCRIPTION

The present invention is concerned with stabilized formulations of IgA, particularly formulations for oral administration, or alternatively, other routes that bring the drug into the site of action (GI tract) such as rectal, intravenous and subcutaneous. The formulations generally comprise the biotherapeutic protein (e.g., IgA) dispersed in a pH buffering agent at a pH of from about 5 to about 8, along with a non-ionic surfactant and one or more optional stabilizing agents.

Formulations according to embodiments of the invention can be used to stabilize monomeric IgA, dimeric IgA, sIgA, glycosylated or non-glycosylated forms of IgA, chemical variants, recombinant forms, minor mutants thereof, or combinations/mixtures thereof. Preferably, monoclonal IgA is used in the formulation. Unless otherwise noted, the terms “IgA” or “Immunoglobulin A” are used for ease of reference to encompass any of the foregoing forms. The IgA protein sequence can be modified for targeted use by changing the variable regions of the constituent kappa (κ) light chains and alpha (α) heavy chains to be specific for particular targets. Specific sequences that differ between IgA molecules that have different targets are generally restricted to the complementarity determining regions (“CDR”) of the variable subdomains of the alpha heavy chains and kappa light chains. It is understood that these target-specific sequence differences are distinct from those that distinguish the different allotypes or subtypes of IgA, e.g. IgA1, IgA2 m1, IgA2m2, and IgA2n. The latter sequence differences are located in the constant subdomains of the respective alpha heavy chains.

For example, Ven-alpha (“Ven-A”) is a recombinant human monoclonal sIgA molecule that specifically binds to human TNF-α. Ven-A consists of four specific alpha (α) heavy chains, four specific kappa (κ) light chains, one joining chain and one secretory component chain. Mature Ven-A is composed of 3363 amino acids in total, which contribute approximately 375 kDa to its total molecular mass. Each of the four kappa (κ) light chains is 214 amino acids in length and has a molecular weight (MW) of approximately 23 kDa. Each of the four alpha (α) heavy chains is of allotype α1, is 474 amino acids in length, and has a protein MW of approximately 51 kDa. The joining chain contains 137 amino acids and has a calculated protein MW of approximately 16 kDa. Finally, the Ven-A secretory component chain contains 585 amino acids and has a protein MW of approximately 64 kDa. As each pair of heavy and light chains forms one binding site for TNF-α, the full Ven-A sIgA assembly conceivably binds up to four individual TNF-α monomers. Binding of Ven-A to TNF-α sterically occludes the interface of TNF-α through which it interacts with its cell surface receptors (TNF-α receptors or TNFRs), and thus prevents TNF-α from initiating pro-inflammatory signaling pathways.

Similarly, Ven-beta (“Ven-B”) comprises a set of recombinant human monoclonal sIgA molecules of allotypes IgA1 or IgA2 m1 that specifically target the surface antigen and minor pilus component cfaE of Enterotoxigenic Escherichia coli (ETEC). The different allotypes are characterized by having alpha (α) heavy chain sequences that differ in their antigen-binding regions, as well as having allotype-specific sequence differences. However, all of the Ven-B IgA molecules target the ETEC cfaE antigen protein, which mediates binding between the pathogenic ETEC bacterium and epithelial cells of host (human) intestines, allowing the bacterium to colonize the gut. The individual mature Ven-B types are each composed of 3315-3375 amino acids in total, which contribute approximately 372-377 kDa to their total molecular masses. Each of the four kappa (κ) light chains is 214 amino acids in length and has a molecular weight (MW) of approximately 23 kDa. Each of the four alpha (α) heavy chains is 473-477 (allotype α1) or 462 (allotype α2 m1) amino acids in length and has a protein MW of approximately 50-51 kDa. By definition, the joining and secretory chains are identical to those of VenA or other types of sIgA molecules. Binding of Ven-B to ETEC cfaE is intended to block the attachment of ETEC bacteria to human gut epithelia.

Ven-A and Ven-B are particularly preferred forms of sIgA produced by recombinant DNA technology in a plant expression system, preferably in monocots, and most preferably in rice, barley, wheat, oats, rye, corn (maize), millet, triticale, and sorghum, such as described in U.S. Pat. Nos. 6,642,437 and 6,991,824, incorporated by reference herein. IgA/sIgA expressed in plants, like other recombinant proteins produced in plant expression systems, is likely characterized by being homogenously or uniformly glycosylated, with a simple (uncomplicated) glycosylation profile, as compared to native IgAs and IgAs expressed in mammalian expression systems. Specifically, N-linked glycosylation of plant-expressed recombinant proteins primarily consists of fairly uniform and homogeneous core glycosylation, with relatively small bi- and tri-antennary high-mannose oligosaccharides. Similarly, while mammalian native IgAs have O-glycosylation sites on the alpha-1 allotype heavy chain, recombinant proteins expressed in the plant systems have a characteristic absence of O-glycosylation. Thus, whereas native sIgA or sIgA produced in mammalian cell culture exhibits a complex variety of structurally heterogeneous glycoforms, potentially involving both N-linked and O-linked glycosylation, a uniform, homogeneous core- or simple-N-linked glycosylation pattern would be characteristic of plant-produced sIgA.

In one or more embodiments, raw extracts from such plant expressions systems preferably comprise a mixture of different forms of IgA (e.g., a mixture of recombinant sIgA and monomeric IgA). That is, the four types of chains that assemble into sIgA are present and expressed simultaneously in plant systems used to manufacture recombinant sIgA. That is, the chains are not reconstituted with each other after extraction/purification but rather coexpressed, and IgA and sIgA assembly occur in vivo within the expressing plant cells. As the assembly pathway for sIgA necessarily includes IgA as a precursor, the plant cells express both IgA and sIgA, resulting in a mixture of the forms based on the efficiency of incorporation of joining- and secretory chains into IgA. These IgA forms may be separated from each other during manufacturing and purification, or maintained together to take advantage of characteristics of each species. Such characteristics include different levels of activation of downstream immune pathways and differing interactions with native microbiota, as well as differing interactions with antigens.

Further, the plant-expressed IgAs are accompanied by any number of plant host proteins characteristic of the particular host expression system. An area of substantial interest in the manufacture of immunoglobulins, including sIgA and IgA, is the control of host cell (expression-system-specific) proteins (HCP) during the manufacturing process. There are increasing efforts to control the exact amounts and identities of HCP in the final purified and formulated immunoglobulin product. Plant-produced sIgAs are also characterized by the presence of plant host/plant expression system-specific HCP, such as seed storage proteins. Other plant components including structural proteins and carbohydrates present at high levels in the plant raw material may also be present in such samples. It is noteworthy that, since grains are classified as GRAS (Generally Recognized as Safe) by the Food and Drug Administration, that such HCPs and other plant host macromolecules do not present a health or safety hazard during oral therapeutic administration.

In contrast, sIgA and IgA recombinantly expressed in animal cell culture systems may be accompanied by host cell proteins, lipids or glycans/oligosaccharides characteristic of these systems, as well as by adventitious pathogens of these systems. In particular, mycoplasma, eukaryotic parasites (for example, T. gondii, T. cruzii, C. parvum, Leishmania sp.) and especially animal viruses (including some highly pathogenic viruses that can infect humans) are known potential contaminants of mammalian cell culture. Such pathogens or their constituents may be present in samples of recombinant sIgA/IgA manufactured from animal cell culture. Native IgA/sIgA obtained directly from the organism, such as from bovine or human colostrum or serum IgA, may similarly be expected to contain HCPs as well as to potentially contain adventitious pathogenic bacteria, parasites, or viruses known to infect the source organism. Animal-origin pathogens include agents implicated in transmissible spongiform encephalopathies (TSEs), also known as prion diseases. TSEs are degenerative brain disorders characterized by tiny holes that give the brain a “spongy” appearance, including Creutzfeldt-Jakob disease (CJD), kuru, fatal familial insomnia, and Gerstmann-Straussler-Scheinker disease (GSS). CJD has been reported in Great Britain and several other European countries and it is believed that this resulted from consumption of beef from cattle with a TSE disease called bovine spongiform encephalopathy (BSE), also known as “mad cow disease.” Other TSEs found in animals include scrapie, which affects sheep and goats; chronic wasting disease, which affects elk and deer; and others. These cases are probably caused by contaminated food/feed. CJD and other TSEs also can be transmitted experimentally to mice and other animals in the laboratory. TSEs are caused by an abnormal version of a protein called a prion. TSE-causing prions may be transmitted through contact with infected tissue, body fluids, or contaminated medical instruments. Normal sterilization does not prevent transmission of TSEs and the prion contamination cannot be detected without a cadaver-derived brain tissue sample. It will be appreciated that plant-expressed IgAs have an advantage of being free of animal components, animal-sourced materials, or any other derivatives of animal origin (such as proteins, metabolic waste products, and zoonotic pathogenic contaminants).

Notwithstanding the foregoing, it will be appreciated that the inventive approaches for stabilizing therapeutic proteins described herein, are also applicable to formulation of IgA and sIgA produced in other expression systems, including CHO, yeast, tobacco, algae, and the like. Moreover, it will be appreciated that the invention applies to formulation of IgA and sIgA for administration against therapeutic targets other than those solely within the GI tract.

Further, it will be appreciated that the underlying (platform) stabilizing formulations described herein, could also be used for other biologic therapeutics such as immunoglobulin G and immunoglobulin M, or any other biologic products that are known to be proteolytically degraded in the gastric and intestinal conditions. Overall, the platform formulations described here provide stability during manufacture, storage, handling, and upon delivery of the administered biological in each of the stomach, small intestine, and colon. The formulations are designed to target diseases, such as by neutralizing an infectious agent or virulence factors, in the intestine and colon and can be taken before and/or after food, preferably in an empty stomach at least 30 min before taking food for better therapeutic effect.

Formulations according to the invention are “stabilized,” which means that the IgA in the formulation remains stable under typical processing, storage, and/or handling stressors, such as mechanical stress, thermal stress, and/or freeze-thaw stress. In general, the formulations will remain stable under long-term storage at a specific temperature; after freeze-thaw cycle; and/or after agitation. In general a “stable” protein is one that shows minimal (or no) changes in secondary and tertiary structure, minimal (or no) degradation or aggregation, minimal (or no) signs of fragmentation, and/or chemical modifications (e.g., oxidation, reduction, deamidation), etc. and maintains integrity of protein primary structure, such that it retains its physical and chemical stability as well as biological activity during storage, even when subjected to stressors. In one or more embodiments, the stability criteria applied here mean that the IgA in the formulation does not change more than 60%, preferably not more than 50%, and most preferably not more than 20% of its total mass, as compared to its initial mass before being subjected to the storage, freeze-thaw cycle, or agitation stress test. In other words, preferably no more than 60%, preferably no more than 50%, and more preferably no more than 20% of the protein in the formulation is degraded during storage and stress conditions as measured by HPLC.

The term “oral stability,” as used herein is further specific to oral/rectal stability upon administration, that is, stability under GI conditions. For ease of reference, “oral” stability is generally referred to herein, and encompasses rectal administration modes, unless expressly noted otherwise. In particular, “orally stable” formulations herein refer to a formulation that demonstrates stability in simulated gastric fluids (SGF) for at least 15 minutes, and in simulated intestinal fluids (SIF) for at least 30 minutes, when incubated at 37° C. In general, an orally “stable” protein is one that shows minimal (or no) changes in secondary and tertiary structure, minimal (or no) degradation or aggregation, minimal (or no) signs of fragmentation, and/or chemical modifications (e.g., oxidation), etc. and maintains integrity of protein primary structure, such that it retains its physical and chemical stability as well as biological activity in vivo, as measured using physiologically relevant in vitro conditions, including SGF and SIF conditions described herein. For oral stability testing in SGF and SIF, degradation can be monitored using size exclusion chromatography. In one or more embodiments, the stability criteria applied here for “oral stability” means that the percentage of intact IgA recovered after digestion (as tested by exposure to SGF and SIF conditions) is greater than 35%, preferably greater than 55%, and more preferably greater than 80%, as compared to the starting amount of IgA. The percent recovery can be measured using standard analytical techniques for protein quantification including but not limited to chromatography, mass spectrometry, spectroscopy, biolayer-interferometry, electrophoresis, immunoassay, or other in vitro and in vivo assays. In other words, preferably no more than 65%, preferably no more than 45%, and preferably no more than 20% of the protein in the formulation is degraded during digestion as measured using SGF and/or SIF in vitro tests.

In one or more embodiments, stabilized formulations according to the invention comprise IgA dispersed in a pharmaceutically-acceptable pH buffering agent at a pH of from 5 to 8. As used herein, the term “pharmaceutically acceptable” means not biologically or otherwise undesirable, in that it can be administered to a subject without excessive toxicity, irritation, or allergic response, and does not cause unacceptable biological effects or interact in a deleterious manner with any of the other components of the composition in which it is contained. A pharmaceutically-acceptable pH buffering agent would naturally be selected to minimize any degradation of the IgA or other agents and to minimize any adverse side effects in the subject, as would be well known to one of skill in the art. Pharmaceutically-acceptable ingredients include those acceptable for veterinary use as well as human pharmaceutical use, and will depend on the route of administration (oral, rectal or other).

Preferred pH buffering agents for use in the stabilizing formulations are those with a buffering capacity in the target pH range of from about 5 to about 8 (e.g., compounds with a pKa of 5 to 8), preferably about 5.5 to about 7.5, more preferably about 5 to about 7, even more preferably about 6 (+/−0.2). Thus, the stabilizing formulations will have a pH of from about 5 to about 8, preferably about 5.5 to about 7.5, more preferably about 5 to about 7, even more preferably about 6 (+/−0.2). In one or more embodiments, the stabilized formulations will have a pH of between 5 and 6. In one or more embodiments, the stabilized formulations will have a pH of about 7 (+/−0.2). In one or more embodiments, the stabilized formulations will have a pH of about 8 (+/−0.2). The pH of the formulations can be adjusted using any pharmaceutically-acceptable acidifying agent or base, such as phosphoric acid or potassium hydroxide and the like, as well as by increasing the concentration of the buffer system.

Suitable pH buffering agents are selected from the group consisting of potassium phosphate, citrate, histidine, acetate, (sodium) bicarbonate, and combinations thereof, with potassium phosphate and histidine being particularly preferred. Buffer concentration in the stabilized formulations can vary, depending upon the selected buffer, other components in the formulation, and their relative concentrations. It will be appreciated that lower buffer concentrations may be required to impart stability and maintain the pH in the target range when one or more additional stabilizing agents are included in the formulation. A broadly contemplated buffer concentration is from about 10 mM to about 1,000 mM. Preferred formulations comprise a buffer concentration of at least about 50 mM, preferably at least about 100 mM, more preferably from about 100 mM to about 500 mM, even more preferably from about 100 mM to about 300 mM. In one or more embodiments, the stabilized formulation comprises a single buffer. In one or more embodiments, mixtures of buffers can be used. pH buffering agents can be prepared by dissolving the compound (typically in base form) in purified water (e.g., distilled water, deuterated water, ultrapure water, deionized water) and titrating the pH down to the designated value with the appropriate acid. pH buffering agents are also commercially available and/or can be stored in liquid solution or as a dried compound that can be reconstituted for use when needed.

In one or more embodiments, the initial stabilized formulation comprises at least one pH buffering agent, and a small amount of non-ionic surfactant for physical stability, to which other stabilizing agents can be added, along with IgA. Exemplary non-ionic surfactants for use in the formulations are polysorbates, such as those selected from the group consisting of polysorbate 80 (polyoxyethylene (20) sorbitan monooleate), polysorbate 20 (polyoxyethylene (20) sorbitan monolaurate), and combinations thereof, in an amount sufficient to physically stabilize the compositions and avoid precipitation or separation of ingredients. In particular, the non-ionic surfactant is included at low levels to reduce non-specific interactions and aggregation of the therapeutic protein in the formulation and adjust surface charges to increase solubility. When present, non-ionic surfactants are used in formulations at a level of less than 1% (w/v), and preferably less than 0.5% (w/v). More preferably, non-ionic surfactant levels in the formulation range from about 0.01% (w/v) to 0.5% (w/v), more preferably from about 0.025% (w/v) to about 0.2% (w/v), more preferably from about 0.03% (w/v) to about 0.1% (w/v), and even more preferably about 0.05% (w/v) (+/−0.02%).

Advantageously, the stabilized formulations will further comprise at least one stabilizing agent selected from the group consisting of: amino acids, sugars/polyols, chloride salts, carboxylic acids, detergents, natural proteins, protein expression extracts, and mixtures thereof. In one or more embodiments, it is preferred that the stabilizing agents are from either plant, mammalian, yeast, or fungal origin. In one or more embodiments, preferred formulations are free of components of insect origin.

Exemplary amino acids for use in the formulations are selected from the group consisting of L-glutamine, glycine, lysine, L-arginine, and combinations thereof, with L-glutamine and glycine being particularly preferred. When present, amino acids are used in the formulation at a level of up to about 500 mM, preferably from about 50 mM to about 500 mM, and more preferably from about 50 mM to about 100 mM.

Exemplary sugars/polyols for use in the formulations are selected from the group consisting of sorbitol, mannitol, trehalose, and combinations thereof. When present, sugars/polyols are used in the formulation at a level of up to about 10% weight by volume (w/v), from about 1% (w/v) to about 10% (w/v) and more preferably from about 5% (w/v) to about 10% (w/v). In one or more embodiments, the formulation is preferably substantially free of sucrose.

Exemplary chloride salts for use in the formulations are selected from the group consisting of sodium chloride, magnesium chloride, potassium chloride, calcium chloride, and combinations thereof. When present, monovalent chloride salt is used in the formulation at a level of up to about 150 mM, preferably from about 50 mM to about 150 mM, and more preferably from about 50 mM to about 100 mM. When present, divalent chloride salt is used in the formulation at a level of up to about 15 mM, preferably from about 5 mM to about 15 mM, and more preferably from about 5 mM to about 10 mM.

Exemplary carboxylic acids for use in the formulations are selected from the group consisting of succinate, lactic acid, malic acid, and combinations thereof. When present, carboxylic acids are used in the formulation at a level of up to about 150 mM, preferably from about 50 mM to about 150 mM, and more preferably from about 50 mM to about 100 mM. In one or more embodiments, the formulation is preferably substantially free of tartrate.

In one or more embodiments, one or more additional stabilizing agents can be used to augment stability against proteolysis, such as for orally administered formulations, as follows.

Exemplary detergents for use in the formulations are zwitterionic detergents, such as sulfobetaines selected from the group consisting of caprylyl sulfobetaine, lauryl sulfobetaine, myristyl sulfobetaine, stearyl sulfobetaine, and combinations thereof. When present, detergents are used in the formulation at a level of at least 0.8% (w/v), preferably from about 0.8% (w/v) to about 2% (w/v). In one or more embodiments, the formulation is preferably substantially free of pluronic F68.

Exemplary natural proteins for use in the formulations include albumin, α-lactalbumin, casein, whey, lactoferrin, lysozyme, tryptone, and combinations thereof. When present, natural proteins are used in the formulation at a level of at least 1% w/w, preferably from about 1% w/w to about 99% w/w, more preferably from about 60% w/w to about 96% w/w and even more preferably from about 80% w/w to about 96% w/w.

In one or more embodiments, protein extracts from the host expression system are included in the formulation as oral stabilizing agents. That is, instead of (or in addition to) purified IgA, the formulation includes the host expression system extract liquid, which contains expressed IgA (among other constituents) extracted from the expression system. In one or more embodiments, plant-based expression systems are used to produce the IgA, such as cereals including wheat (Triticum sps.), rice (Oryza sps.), barley (Hordeum sps.), oats (Avena sps.), rye (Secale sps.), corn (maize) (Zea sps.), and millet (Pennisettum sps.), triticale, and sorghum (Sorghum bicolor).

Exemplary protein extracts for use in the formulations may be derived from various plant tissue including seed, grain, leaves, roots, stems or fruits expressing IgA. Other expression systems include algae, yeast, CHO (Chinese hamster ovary cell line), and the like, which can be engineered to express IgA. Protein expression extracts from these systems will include other constituents, including proteins, fats, starches/carbohydrates, fibers, etc. endogenous to the host system. Without wishing to be bound by theory, it is believed these other constituents when included in the formulation in their unpurified form provide an additional layer of protection for IgA against degradation under gastric and intestinal conditions.

Regardless of the expression system used, the resulting expression product is extracted from the host expression system for use in the formulation using suitable techniques for each type of expression system. It will be appreciated that extraction techniques are preferably carried out under non-denaturing conditions so that damage to the expressed protein(s) is minimized. Solvents can be used for solubilization and/or extraction of the protein without loss of activity. In the case of plant-based expression systems, cells transformed as above are used to regenerate plants, and plants are allowed to mature, through cultivation practices. Consequently, heterologous/recombinant biotherapeutic proteins are produced in the plant tissue. Then the plant tissue can be processed, if necessary, to facilitate extraction of the proteins. These proteins, can be extracted under defined extraction conditions which may include denaturing and non-denaturing conditions. Plant processing procedures can include grinding, filtration, heat, pressure, salt extraction, evaporation, and the like. For example, parts of the host expression system plant materials can be mechanically processed to form a flour, powder, paste, meal, or other pulverized form which is then contacted with an aqueous and/or organic extraction fluid. Optionally, the extract can be further treated to partially concentrate the extract and/or remove unwanted components. In a preferred method, grains or seeds, such as rice seeds are processed or milled to a flour, powder, paste, or homogenate, and the resulting processed host expression system raw material is then suspended in an extraction medium or extraction media, such as aqueous solutions, phosphate buffered saline, ammonium bicarbonate buffer, ammonium acetate buffer, Tris buffer, acetic acid buffer, chloride salt solution, ammonium bicarbonate, ammonium acetate, and the like.

In one or more embodiments, the expression system extract is not purified (e.g., via chromatography) for use in the formulations; however, it may be concentrated (e.g., by centrifugation) or otherwise filtered by gross filtration (˜20 μm down to 0.2 μm) methods, if desired. In general, using rice expression systems described herein, about 0.7% of the resulting extract comprises proteins (including IgA). Thus, in the formulations, aside from proteins, the extract itself is present in the formulations at a level of up to about 99.3% w/w, preferably from about 60% w/w to about 99.3% w/w, and more preferably from about 80% w/w to about 99.3% w/w.

The formulation is prepared by dispersing the IgA in the pH buffering agent. In one or more embodiments, the extracted IgA may be first separated from its extraction medium, such as by repeated dilution and concentration with the selected buffering system to isolate the IgA for formulating. The IgA may be further purified before using in the formulation. Further, the IgA may be lyophilized and reconstituted before using in the formulation. IgA may also be stored in liquid solution, followed by thawing before using in the formulation. Regardless, the IgA is dispersed in the pH buffering agent at the selected pH value along with a non-ionic surfactant.

One or more additional stabilizing agents, as described herein, may be dispersed in the formulation with the IgA.

The formulation can be prepared according to a suitable format for the desired route of administration, and may be a liquid suspension and/or dried (lyophilized) powder, among other forms. Exemplary administration forms include hard- or soft-shelled powder-filled uncoated, or enteric-coated capsules, dissolving tablets, caplets, free or encapsulated minitablets, multi-particulates, lozenges, pastilles, granules, microspheres, nanoparticles, injectable liquid solution, liquid bolus, oral solutions, oral suspensions, syrup, elixirs, gel, bulk emulsion, nebulized mist, aerosols, micro- or nano-emulsions, liposomes, or suppositories, and the like. In addition, these formulations can be prepared as a powder to be suspended with liquid upon administration.

These formulations maybe directly administered, or can also be prepared to be added to the food or drink of the subject, such as a food supplement, for example in infant formula or child nutrition drink.

In addition, a formulation described here can comprise pharmaceutically acceptable preservatives/antibacterials (e.g., benzoic acid), antioxidants, flavoring agents, colorants, chelators, sweeteners, suspending agents, diluents, glidants, lubricants, and the like. Further, the formulation may be dispersed or suspended or compacted with a number of inert carriers, fillers, or bulking agents, such as starch, cyclodextrins, salts, osmolytes, metals, and the like.

The formulations may contain the single biotherapeutic protein (e.g., IgA), or may include a combination of biotherapeutic proteins, if desired. Further, additional therapeutic and/or prophylactic agents could be included with the formulation as part of a combination therapy.

In one or more embodiments, the stabilized formulations remain stable during manufacturing, purification, and storage, and have at least 50% recovery of stable IgA (as compared to a starting IgA level), after being subjected to a stressor, such as agitation and/or freeze-thaw.

In one or more embodiments, the stabilized formulations remain stable and have at least 35% recovery of stable IgA (as compared to a starting IgA level), after being subjected to GI conditions (e.g., either SGF and/or SIF).

In one or more embodiments, an exemplary stabilized formulation comprises (consists essentially or even consists of) IgA, histidine pH buffering agent, polysorbate-80, and potassium chloride.

In one or more embodiments, an exemplary stabilized formulation comprises (consists essentially or even consists of) IgA, potassium phosphate pH buffering agent, L-glutamine, sorbitol, sodium chloride, and succinate.

In one or more embodiments, an exemplary stabilized formulation comprises (consists essentially or even consists of) IgA, potassium phosphate pH buffering agent, sorbitol, sodium chloride, and succinate.

In one or more embodiments, an exemplary stabilized formulation comprises (consists essentially or even consists of) IgA, potassium phosphate pH buffering agent, sorbitol, and sodium chloride.

In one or more embodiments, an exemplary stabilized formulation comprises IgA, optional potassium phosphate pH buffering agent, and from about 0.025% (w/v) to about 0.2% (w/v) polysorbate 80, preferably from about 0.05% to about 0.1% polysorbate 80, and more preferably from about 0.05% (w/v) to about 0.025% (w/v) polysorbate 80.

In one or more embodiments, an exemplary stabilized formulation comprises (consists essentially or even consists of) IgA, potassium phosphate pH buffering agent, polysorbate 80, and α-lactalbumin.

In one or more embodiments, an exemplary stabilized formulation comprises (consists essentially or even consists of) IgA, potassium phosphate pH buffering agent, polysorbate 80, and Myristyl Sulfobetaine.

In one or more embodiments, an exemplary stabilized formulation comprises (consists essentially or even consists of) IgA in host expression extract, potassium phosphate or histidine pH buffering agent, and polysorbate 80 (˜0.05% w/v).

In one or more embodiments, an exemplary stabilized formulation comprises (consists essentially or even consists of) IgA, optional histidine pH buffering agent, and from about 0.025% (w/v) to about 0.2% (w/v) polysorbate 80, preferably from about 0.05% to about 0.1% polysorbate 80, and more preferably about 0.05% (w/v) polysorbate 80. Preferably about 1000 mM Histidine pH 6.0 and about 0.05% (w/v) polysorbate-80.

In one or more embodiments, an exemplary stabilized formulation comprises (consists essentially or even consists of) IgA and a (sodium) bicarbonate buffering agent, up to two grams per dose.

Regardless of the embodiment, the stabilized formulations will comprise a therapeutically effective amount of IgA dispersed in the formulation. In some embodiments, the formulations will comprise up to about 200 mg/ml IgA, preferably up to about 170 mg/ml IgA, more preferably from about 1 mg/ml to about 200 mg/ml IgA described herein, and even more preferably from about 1 mg/ml to about 100 mg/ml IgA. As used herein, the term “therapeutically effective” refers to the amount and/or time period that will elicit the biological or medical response of a tissue, system, animal, or human that is being sought by a researcher or clinician, and in particular elicit some desired therapeutic effect. For example, in one or more embodiments, therapeutically effective amounts and time periods are those that deliver an effective amount of IgA representing a daily dose of from about 10 μg per kilogram of bodyweight to about 100 mg per kilogram bodyweight in the patient. One of skill in the art recognizes that an amount or time period may be considered “therapeutically effective” even if the condition is not totally prevented or eradicated but improved partially.

In some embodiments, the formulation is substantially free of any other ingredients except those noted, where the term “substantially free” means that the ingredient is not intentionally added or part of the formulation, although it is recognized that residual or incidental amounts or impurities may be present in low amounts (e.g., less than about 0.1% by weight and preferably less than about 0.01% by weight, based upon the total weight of the formulation taken as 100% by weight). For example, in one or more embodiments, stabilized formulations are substantially free of protease/peptidase inhibitors, such as trypsin inhibitor, puromycin dihydrochloride, aprotinin, and N-acetyl cysteine. In one or more embodiments, stabilized formulations are substantially free of animal-sourced components.

Formulations according to the embodiments disclosed herein are useful in treating, mitigating and/or preventing a variety of diseases and/or conditions that can be treated using antibody-based therapy, such as immune deficiencies, systemic conditions, inflammation or disorders affecting mucosal membranes, cardiovascular conditions, metabolic syndrome, obesity, osteoporosis, neuropathies, cancers, infectious diseases (bacterial, viral or fungal), gastrointestinal disorders, microbiome-mediated health conditions, and conditions such as chronic or acute diarrhea, Crohn's, colitis, celiac disease, inflammatory bowel disease, infectious diseases and the like. The formulations can also be used as immunoglobulin supplements, such as colostrum supplements for infant and newborn care, such as in infant formula. Immunoglobulin supplements are also useful to address leaky gut, or part of a probiotic formulation to treat, repair or establish/re-establish the microbiome in a subject. Thus, embodiments described herein have therapeutic and/or prophylactic uses, and in particular can be used for prophylactic treatment of various conditions mediated by immunoglobulin, and particularly IgA, deficiency.

In general, the stabilized formulations are administered prophylactically, that is, before the subject demonstrates observable clinical symptoms of the disorder. Alternatively, the stabilized formulations can be administered to a subject that is already exhibiting observable clinical symptoms of the disorder. In either case, these stabilized IgA formulations can be used to reduce the incidence or severity of clinical symptoms and/or effects of the disorder, and/or reduce the duration of the symptoms/effects in the subject.

The methods comprise orally or rectally administering the stabilized formulation to a subject in need thereof. Other suitable routes of administration include parenteral and mucosal membrane delivery methods as well as systemic routes of administration or direct injection or application into/on a tissue region of the subject, including, without limitation, sublingual, topical, nasal, buccal, ocular, vaginal, inhalation, intravenous, subcutaneous, intramuscular, infusion.

The formulations advantageously remain stable during processing, compounding, packaging, storage, shipping, and administration, for delivery of the active form of the IgA to the subject. The formulations were specifically designed to be stable when subjected to stress conditions such as shaking or agitation, high temperature, and freeze-thaw cycles. The formulations can be used in processing and manufacturing of the active ingredient and the final finished drug product for the treatment of patients as well as in pre-clinical and clinical studies.

The formulations are preferably stable under storage conditions at about 25° C. and 60% RH for up to about 6 months, at about 40° C. and 60% RH for up to about 3 months, at about 4° C. for at least about 12 months, at about −20° C. for at least about 24 months, and at about −80° C. for at least about 60 months.

The oral formulations advantageously remain stable during digestion (e.g., under gastric and intestinal conditions), for delivery of the active form of the IgA to the GI tract of the subject.

These formulations were specifically designed to be stable under such conditions.

Additional advantages of the various embodiments of the invention will be apparent to those skilled in the art upon review of the disclosure herein and the working examples below. It will be appreciated that the various embodiments described herein are not necessarily mutually exclusive unless otherwise indicated herein. For example, a feature described or depicted in one embodiment may also be included in other embodiments, but is not necessarily included. Thus, the present invention encompasses a variety of combinations and/or integrations of the specific embodiments described herein.

As used herein, the phrase “and/or,” when used in a list of two or more items, means that any one of the listed items can be employed by itself or any combination of two or more of the listed items can be employed. For example, if a composition is described as containing or excluding components A, B, and/or C, the composition can contain or exclude A alone; B alone; C alone; A and B in combination; A and C in combination; B and C in combination; or A, B, and C in combination.

The present description also uses numerical ranges to quantify certain parameters relating to various embodiments of the invention. It should be understood that when numerical ranges are provided, such ranges are to be construed as providing literal support for claim limitations that only recite the lower value of the range as well as claim limitations that only recite the upper value of the range. For example, a disclosed numerical range of about 10 to about 100 provides literal support for a claim reciting “greater than about 10” (with no upper bounds) and a claim reciting “less than about 100” (with no lower bounds).

### EXAMPLES

The following examples set forth methods in accordance with the invention. It is to be understood, however, that these examples are provided by way of illustration and nothing therein should be taken as a limitation upon the overall scope of the invention.

### Materials and Methods

### Immunoglobulin A

Experiments were conducted using plant-expressed IgAs from the ExpressTec™ platform of Ventria Bioscience, which are free of contamination from any by-products of animal, human, or microbial origin.

### Size Exclusion Chromatography (SEC)

Experiments were conducted using an Agilent 1290 Infinity II LC System equipped with a temperature-controlled autosampler adjusted to 4° C. and set to detection wavelengths 280 nm and 215 nm. An Agilent AdvanceBio SEC 300 Å, 2.7 μm, 7.8×300 mm column with a corresponding AdvanceBio SEC 300 Å, 7.8×50 mm, 2.7 μm LC guard column was used for characterization. Initially, the column was equilibrated for 20 CV with a mobile phase containing 150 mM sodium phosphate, pH 7.0 and a constant flow rate of 0.2 mL/min. The column oven temperature was set to 25° C. Samples prepared at 1 mg/mL were injected in a volume of 10 and the run was monitored for 30 min. Using a similar method and column, samples were also analyzed with a mobile phase containing 1% trifluoroacetic acid (TFA), 1% formic acid, 20% Acetonitrile (ACN). The data were processed with Open LAB CDS ChemStation edition Rev.C.01.08 [210] or Rev.C.01.07 SR3 [465].

### Cation-Exchange Chromatography

Experiments were performed using an Agilent 1290 Infinity II LC System equipped with temperature-controlled autosampler adjusted to 4° C. and set to detect wavelengths 280 nm and 215 nm. An Agilent Bio MAb, NPS, 5 μm 4.6×250 mm column with a corresponding Agilent Bio MAb, NPS, 5 μm, non-porous 4.0×10 mm LC guard column was used for characterization. Samples were analyzed with a step gradient from 10 mM sodium phosphate, 10 mM sodium bicarbonate, pH 5.0 (mobile phase A) to 10 mM sodium phosphate, 10 mM sodium bicarbonate, pH 9.5 (mobile phase B). The column was equilibrated for 20 CV with mobile phase A at a flow rate of 0.8 mL/min. A step gradient of mobile phase A was held at 100% for 5 min followed by a decrease from 10% to 0% within 40 min. Finally, 0% was held for 10 min. The column oven temperature was set to 30° C. Samples prepared at 1 mg/mL were injected in a volume of 10 and the run was monitored for 60 min. Data were analyzed using Open LAB CDS ChemStation edition Rev.C.01.08 [210] or Rev.C.01.07 SR3 [465].

### Sample Preparation and Stress Conditions

Formulated samples of 1 mg/ml were prepared from a stock solution by extensive diafiltration and buffer exchange with the desired formulation composition, using Amicon ultra-centrifuge 0.5 ml filters (3000 MWCO, Merck Millipore ltd).

Thermal stress: formulated samples were incubated at 40° C., 60% relative humidity (RH) for 10 days.

Freeze-thaw conditions: formulated samples were subjected to five cycles of freeze-thaw with a hold time of 10 minutes at 37° C. and hold time of 2 hours at −80° C.

Agitation: formulated samples were subjected to agitation on a revolving mixer at 30 rpm for 16 hours at room temperature.

UV-Vis spectrophotometer: Samples were centrifuged at 5000 rpm for 5 min. The absorbance was measured at 280 nm, and the specific absorption coefficient (d) used for sIgA was d^{0.1%}_{280 nm}=1.375 (L×g⁻¹×cm⁻¹). The molecular weight of sIgA is approximately 380 kDa.

High concentration formulations: samples for high concentration formulation studies were prepared at concentrations greater than 5 mg/ml.

### Screening pH Conditions for Ven-A Stable Formulation

Stable formulation development of a biologic therapeutic requires in-depth knowledge of both physicochemical and physiochemical characteristics. It is important to understand the pH range within which the protein of interest is chemically and physically stable, because pH may have profound effects on chemical degradation. Examples of pH-dependent chemical degradation/modifications are deamidation, oxidation, proteolysis, beta-elimination, and disulfide scrambling.

Ven-A was formulated from pH 2 to pH 10 and incubated for 10 days at 40° C. in 60% RH. Samples were analyzed using native SEC-HPLC and denatured SEC-HPLC to compare the effect of pH on the formation of High Molecular Weight Species (HMWS) and Low Molecular Weight Species (LMWS) during incubation. The resulting data were used to identify the most stable pH ranges for formulation development. Cation exchange HPLC (CEX-HPLC) was used to identify acidic and basic species of Ven-A generated due to chemical degradation.

The formulation conditions with the highest Ven-A recovery (%) were selected. Ven-A formulation in 20 mM citrate pH 2, pH 3, and pH 4 resulted in 66%, 39%, and 16% recovery (Total Area Under Curve, TAUC), respectively (FIG. 1). A non-stressed control sample was prepared from the stock solution and was used to prepare samples at different pH conditions. The control sample showed 98% (TAUC) recovery, containing less than 2% of LMWS. Similarly, formulation in 20 mM citrate pH 6, pH 7, and pH 8 led to 75%, 85%, and 85% recovery, respectively (FIG. 1). The data indicated relatively higher chemical stability in the pH range between pH 6 and pH 8. Thus, Ven-A should be formulated in this pH region for optimal chemical stability. Ven-A was also formulated in Tris 20 mM pH 8, pH 9, and pH 10 resulting in recoveries of 85%, 89%, and 89%, respectively (FIG. 1). Overall, data collected from denatured SEC indicated that optimal pH regions for formulation are pH 6, pH 7, pH 8, and pH 9, preferably pH 6, pH 7 and pH 8 (FIG. 1).

Due to the complex nature of proteins, formulation development requires orthogonal analytical techniques for selection and identification of factors that affect stability. Therefore, in addition to denatured SEC-HPLC, native SEC-HPLC was used to screen and identify optimal pH conditions. The control sample (non-stressed) used to prepare formulations at different pH showed 99% monomeric species with <1% LMWS (FIG. 2). After storage at 40° C. and 60% RH, samples formulated at pH 2, pH 3, pH 4, and pH 5 showed 0%, 0%, 6% and 5% monomeric species (i.e., poor formulation pH), respectively (FIG. 2). In contrast, formulations at pH 6, pH 7, and pH 8 showed 85%, 65% and 81% (FIG. 2). Native SEC data demonstrated that pH 6, pH 7, and pH 8 were a good formulation pH conditions (FIG. 2). Formulations at pH 6 and pH 8, possessing the highest area % of the main peak were selected (FIG. 2). Also, formulation in 20 mM Tris pH 9 and pH 10 showed 55% and 50% main peak, respectively. The data demonstrated Ven-A is more stable at pH values above pH 6 rather than at more acidic pH. Overall, formulation pH 6>pH 8>pH 7, listed most stable to relatively less stable.

Ideally, a protein molecule should not be chemically modified at a selected pH for formulation. Before storage in the formulation buffer, a protein molecule can be chemically modified during the manufacturing process. This is not surprising given the myriad of processes the protein might face, such as harsh pH conditions during elution and neutralization in affinity chromatography which ultimately leads to deamidation and fragmentation if not performed in a gentle manner. To gather additional information on modifications that lead to Ven-A degradation, cation exchange chromatography (CEX) was used to monitor acidic and basic species generated after samples were subjected to stress. CEX is a powerful analytical technique that separates acidic and basic species of protein molecules. Acidic species (those eluting prior to the main peak) are more negatively charged (due to deamidation, for example), while basic species (those after the main peak) are more positively charged compared to the main peak. Changes in the charge profile of the main unmodified peak on CEX chromatograms are pH-dependent and correlate with the chemical stability of the protein or the lack thereof. Hence, CEX was used to identify the pH region where there is minimum modification maintaining the highest area % of the main peak (unmodified).

The control sample showed 5% acidic and 6% basic species with the area % of the main peak being 89% of the TAUC (FIG. 3). Chemical modification of the control was presumably generated during manufacturing. In formulations at pH 2 to pH 5 the unmodified peak was absent (0% TAUC, poor formulation pHs), indicating significant degradation and further defining this unstable pH range. The majority of the species observed at pH 2 to pH 5 were acidic species, presumably due to deamidation as a main pathway of degradation. Samples formulated at pH 6 showed 72% main peak (i.e., unmodified), 19% acidic species, and 9% basic species (FIG. 3). These data indicated significant chemical stability at pH 6 compared to lower pH values under selected stress conditions. Similarly, samples formulated at pH 7 showed 58% main peak, 42% acidic, and 0% basic species (FIG. 3). Formulation at pH 7, pH 8, and pH 9 showed 79%, 74%, and 82% Ven-A recovery, respectively, with main peaks mostly unmodified, all of which indicates a relatively stable solution condition. Taken together, these observations indicate Ven-A is chemically stable at pH>6, preferably pH 6.0 and 8 as the optimal formulation condition (pH 6>pH 8>pH 7).

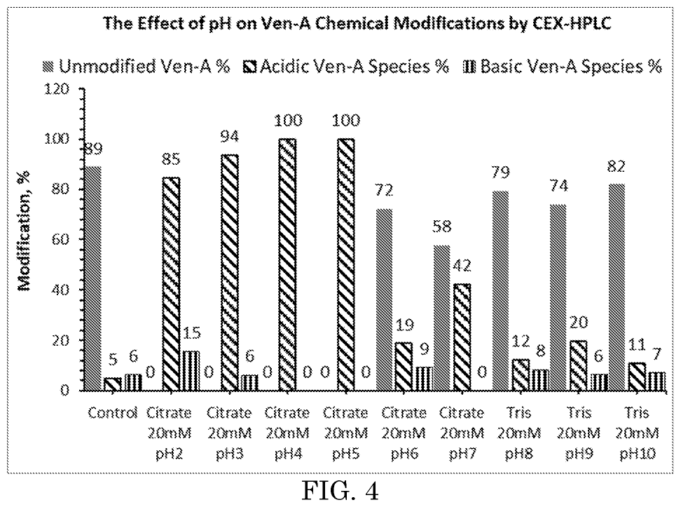

Conformational stability of Ven-A from pH 2 to 10 was evaluated using differential scanning calorimetry (DSC). Solution pH is a known contributor to the stability of tertiary protein conformation, an essential requirement for functional activity. Therefore, monitoring conformational stability is an integral part of formulation development. In general, a pH condition resulting in a lower Temperature of protein denaturation (Tm) value is not preferred for formulation, unless specific attributes such as high stability, solubility, and minimum aggregation are observed using a battery of analytical techniques. No transition was observed at pH 2, indicating a perturbed tertiary conformation (unstable conformation), suggesting pH 2 is unsuitable for formulation. Formulation conditions at pH 3, pH 4, and pH 5 showed Tm equal to 61° C., 67° C., and 71° C., respectively (FIG. 4). A gradually increasing trend in Tm values was noted with increasing pH values, indicating more conformational stability (FIG. 4). At pH 6, pH 7, pH 8 and pH 9 the Tm value remained stable at 72° C. and was preferred for formulation (FIG. 4). This exceptionally similar Tm value demonstrated stable conformation of Ven-A while pH was changing, a condition suitable for formulation development. No thermal transition was observed at pH 10 (poor formulation pH), likely due to perturbed tertiary structure or conformational instability. Although formulations at pH 7 and 8 provide good stability, formulation at pH 6 is preferred (FIG. 4).

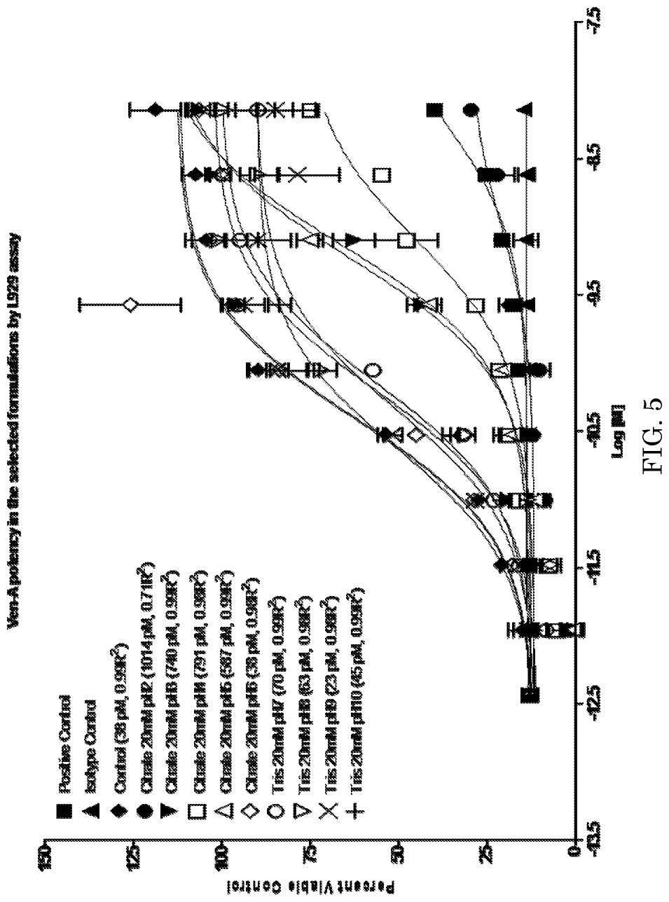

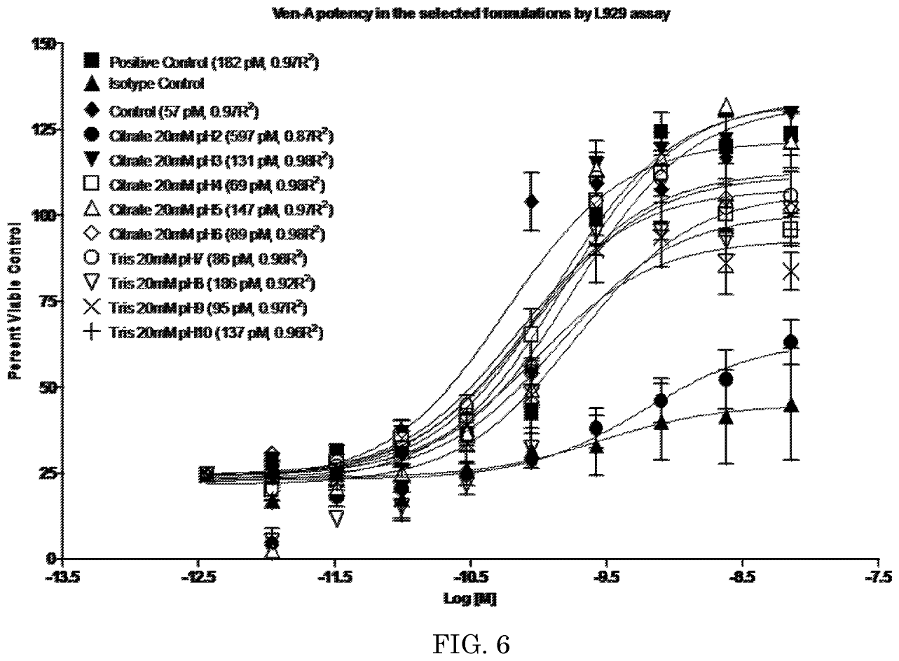

Samples were subjected to stress at 40° C., 60% and tested their functional activity using L929 cell-based bioassay (FIG. 5 and FIG. 6). Beside measuring potency, a bioassay can serve to confirm structural integrity of protein therapeutics. Samples formulated between pH 2 to pH 5 showed low binding affinity suggesting a loss of potency (FIG. 4), likely due to loss of structural integrity as shown from SEC, DSC, and CEX (FIG. 1, FIG. 2, FIG. 3 and FIG. 4). Also, samples formulated above pH 6 showed high binding affinity, suggesting, the structural integrity was maintained after stress condition (FIG. 5 and FIG. 6). Overall, the trend observed in potency assay using murine Tumor Necrosis Factor alpha (mTNF-α) was remarkably similar to the analytical characterization. However, differences were not apparent when the L929 mouse (C3H/An) fibroblast immortalized cell line was incubated with human Tumor Necrosis Factor alpha (hTNF-α) (FIG. 6).

### Screening Buffers for Ven-A Stable Formulation

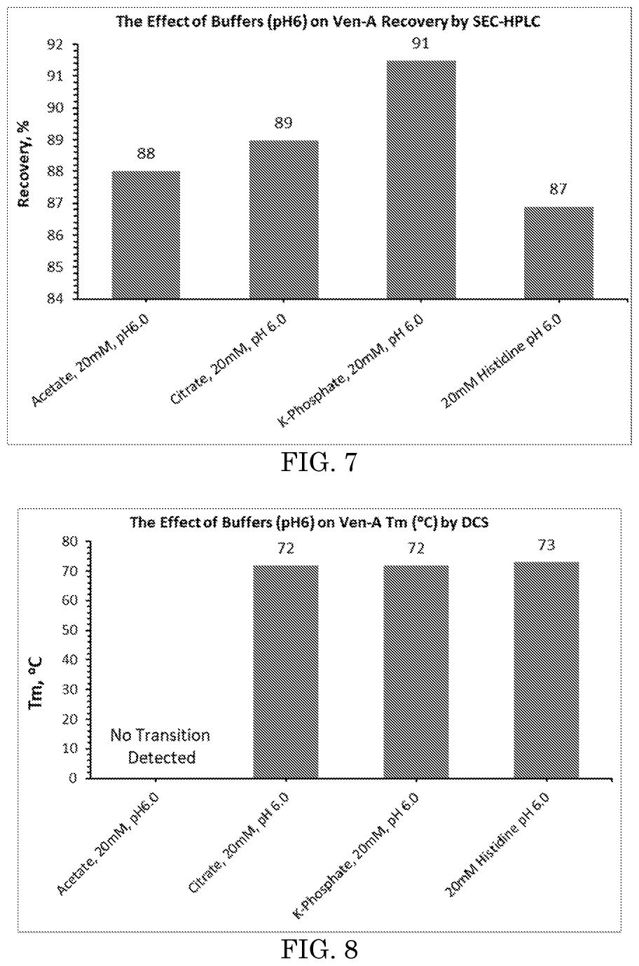

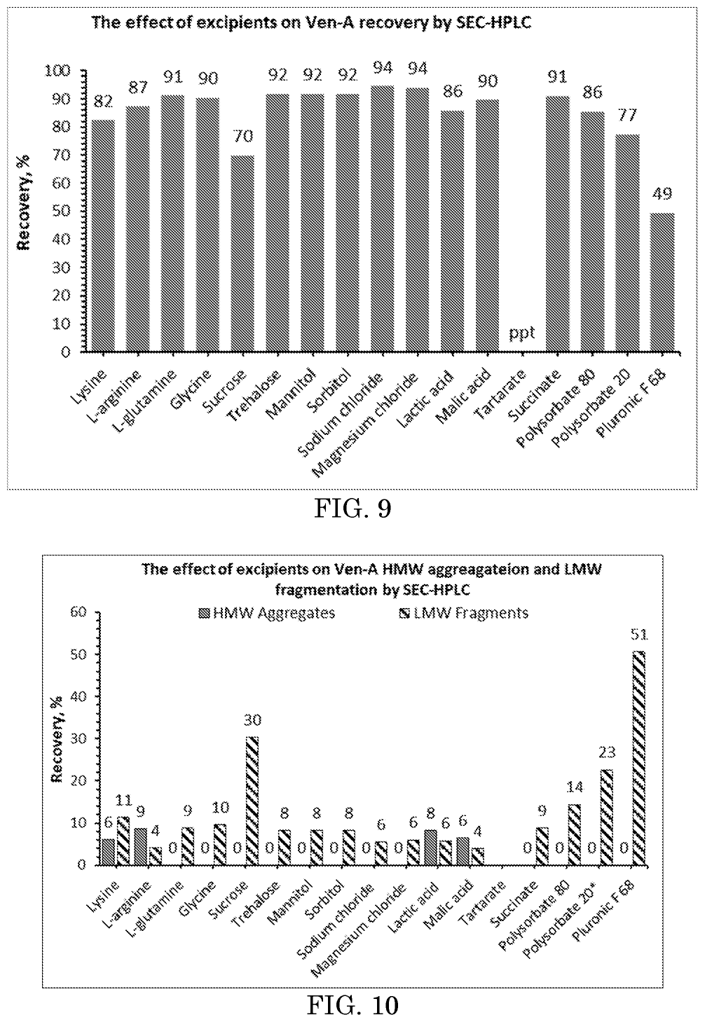

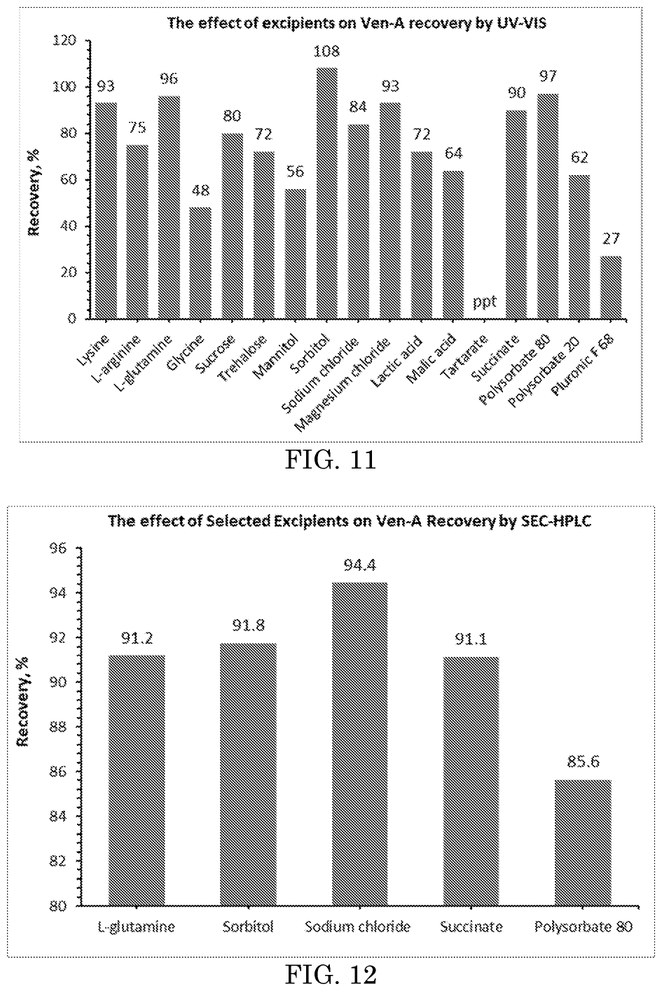

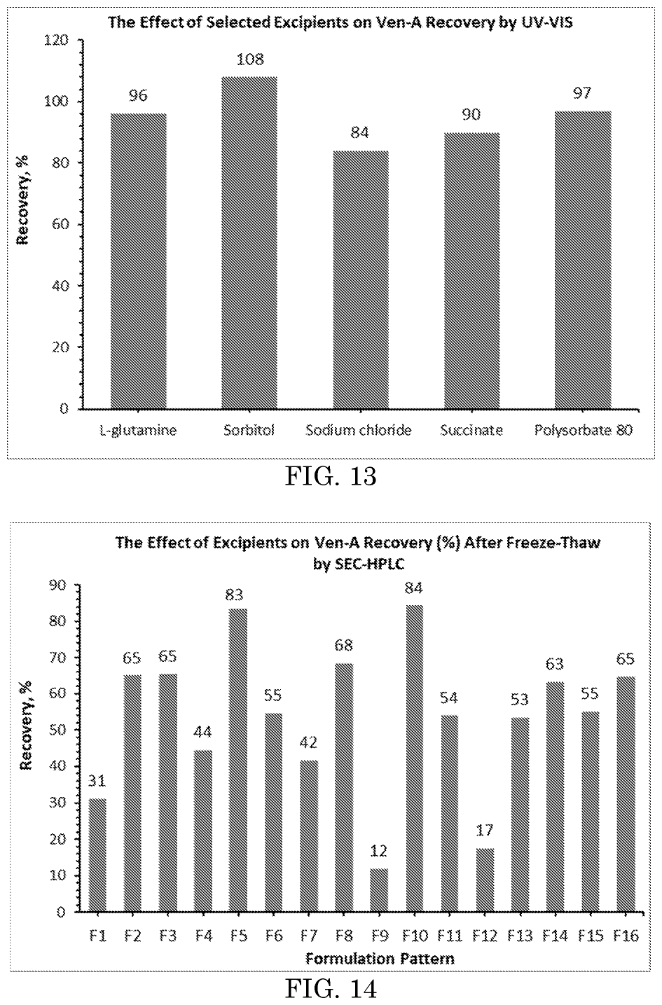

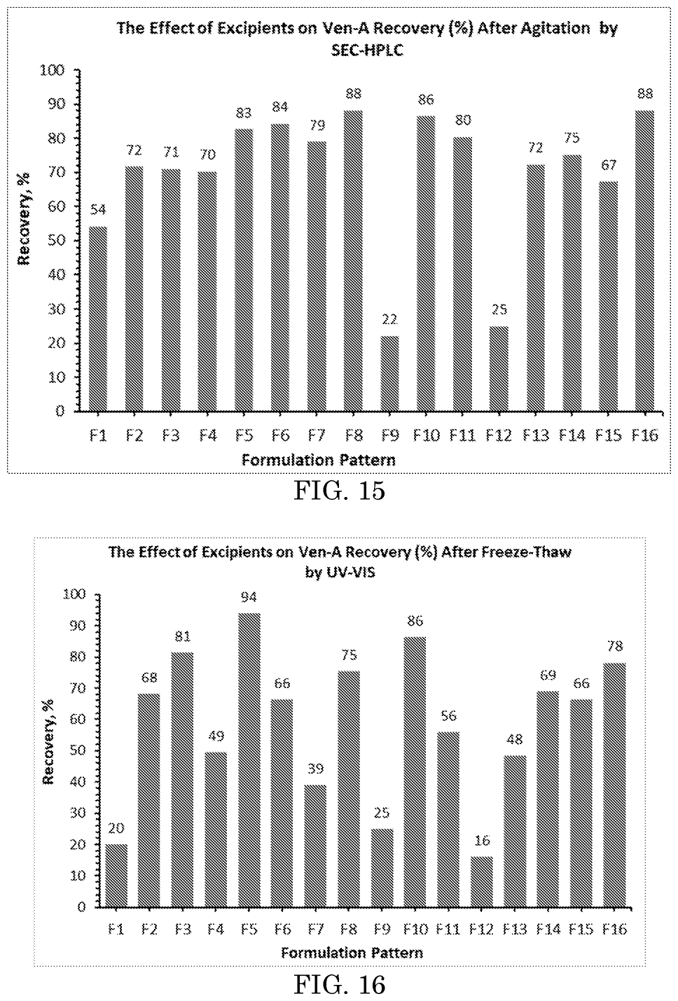

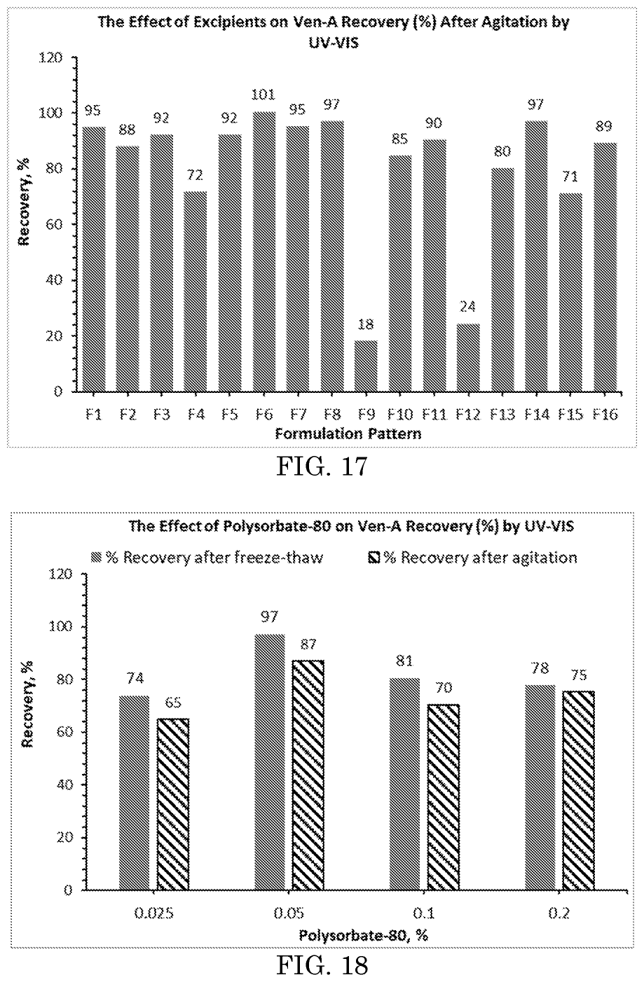

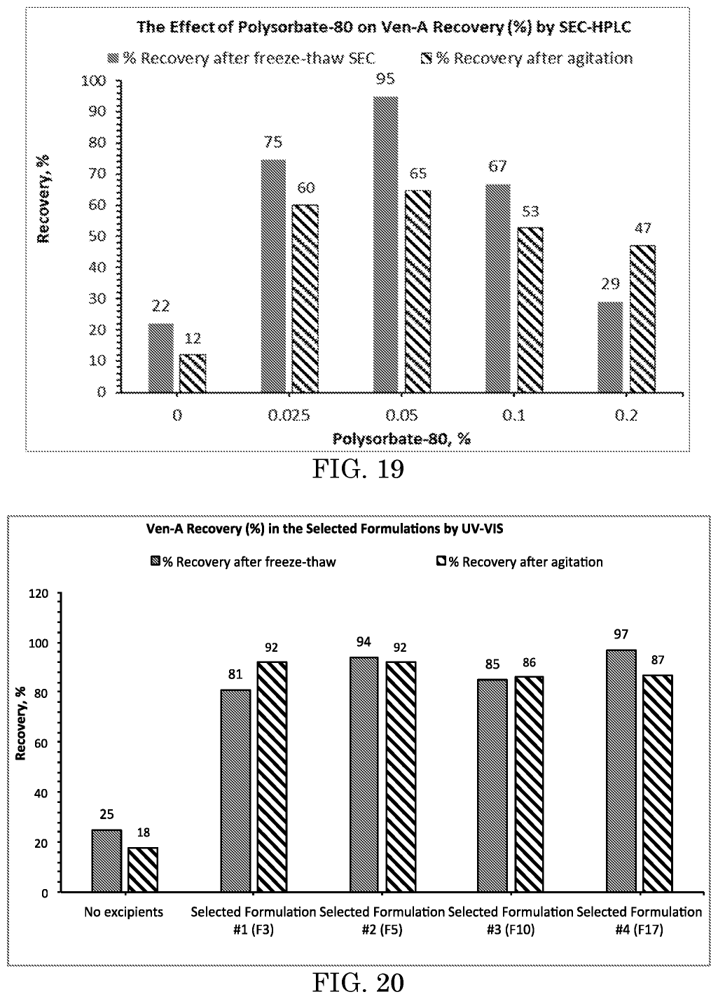

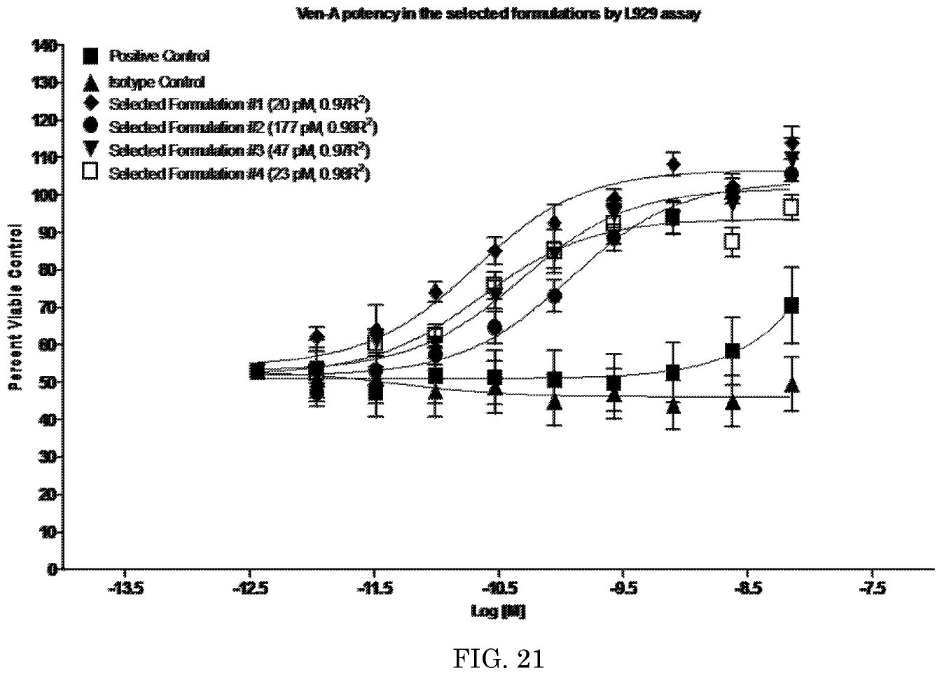

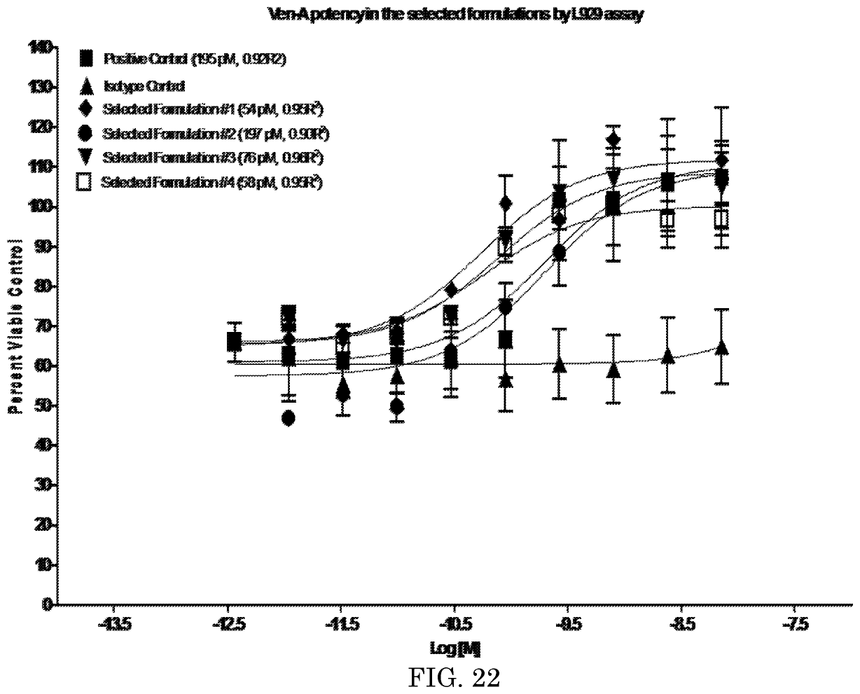

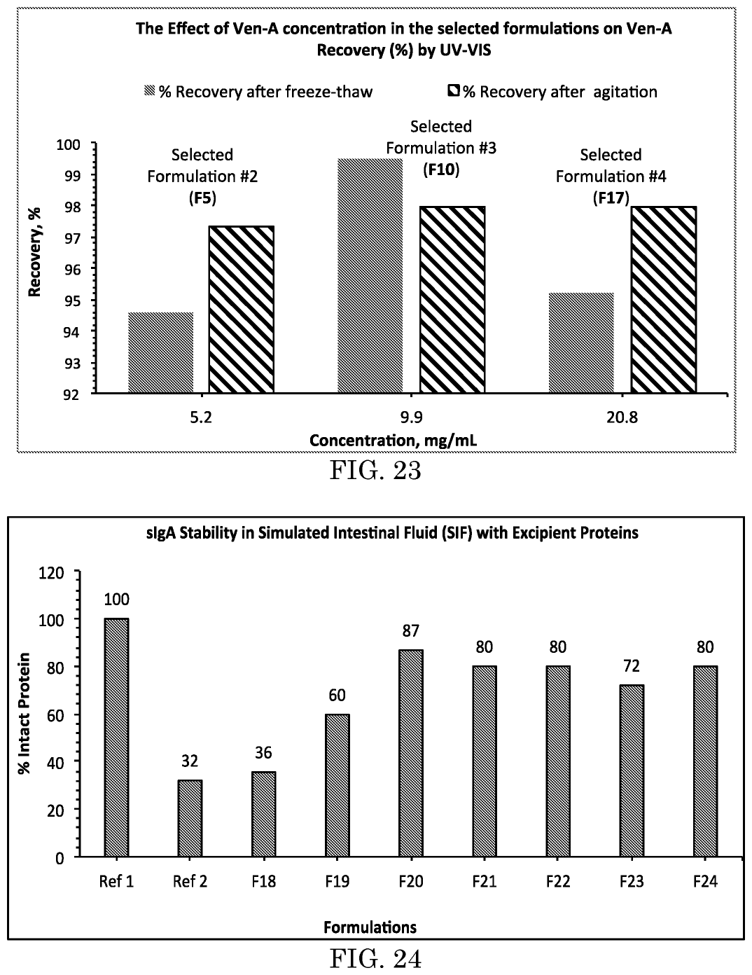

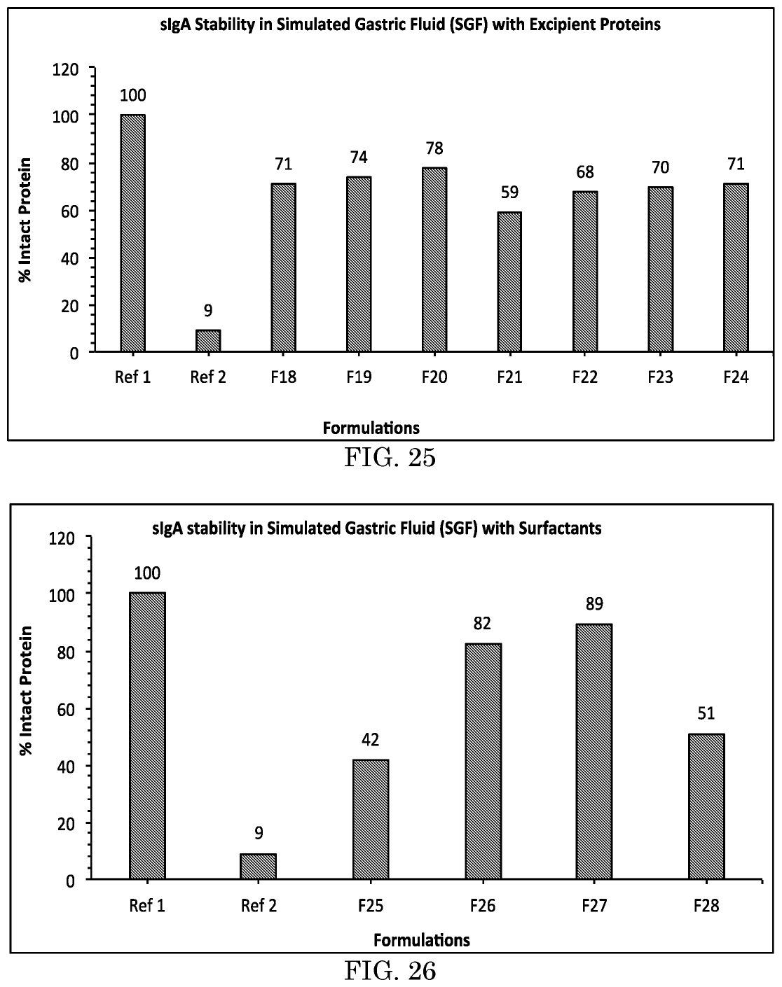

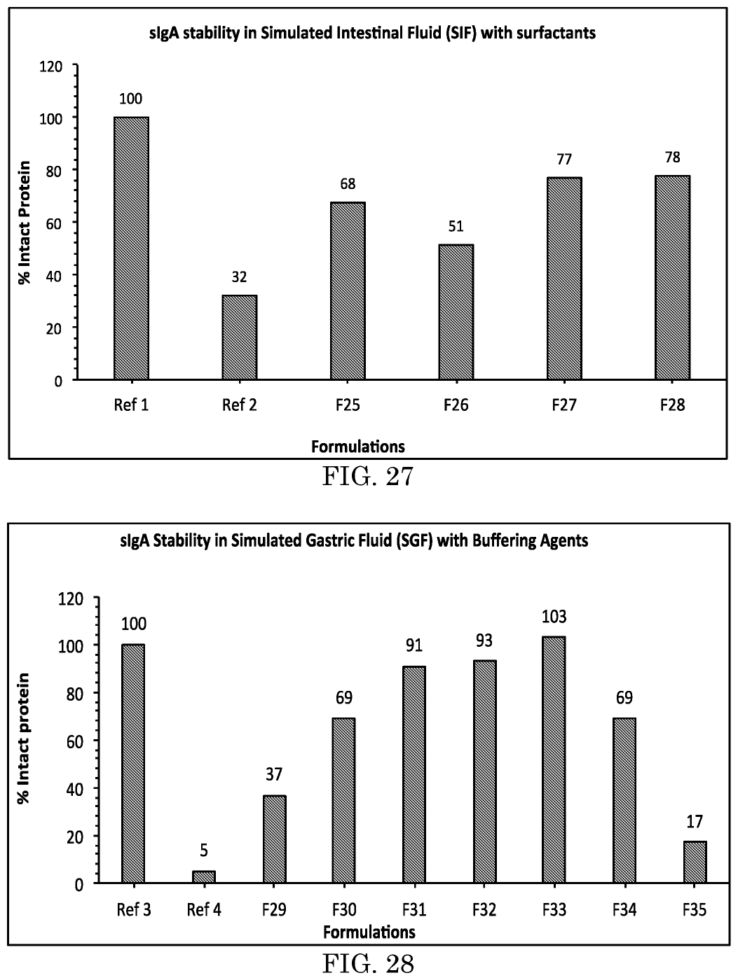

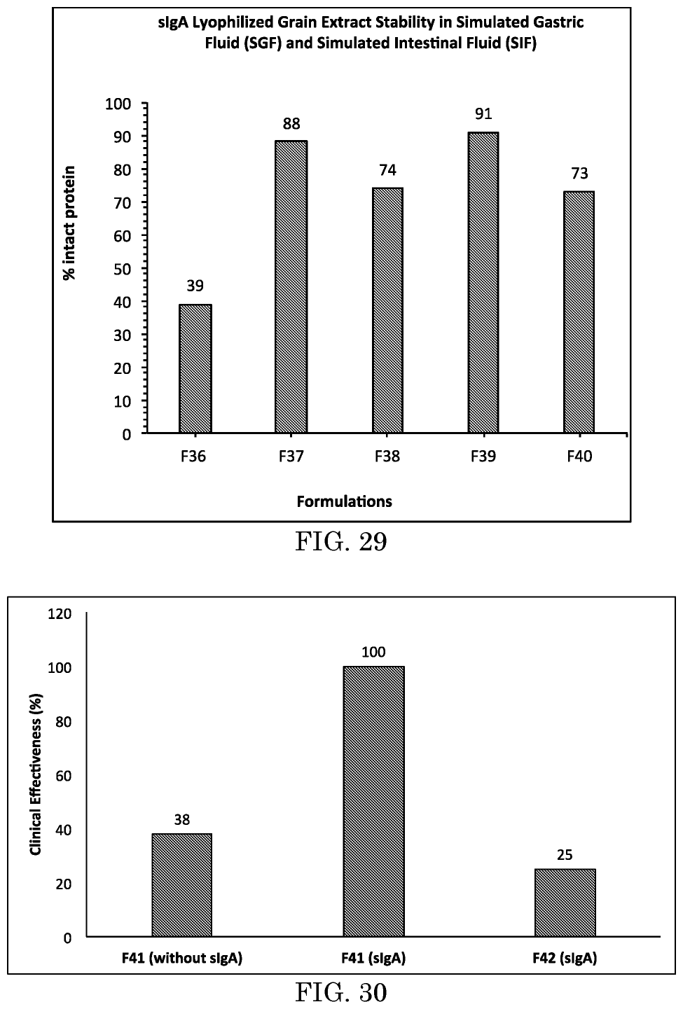

Based on native Size-Exclusion High-Performance Liquid Chromatography (SEC-HPLC), denatured SEC-HPLC, cation-exchange high-performance liquid chromatography. (CEX-HPLC) and Differential Scanning calorimetry (DSC) data, pH 6 was selected for optimal stability (FIG. 1, FIG. 2, FIG. 3, and FIG. 4). The next step in the formulation development was to select a buffer that maintains conformational stability with minimal chemical and physical degradations. Potential formulation buffers at pH 6 include: 20 mM acetate, 20 mM potassium phosphate, 20 mM citrate, and 20 mM histidine. Formulations were subjected to stress at 40° C., 60% RH for 10 days and analyzed using SEC, DSC and inspected visually for solution clarity. This allows selection of potential formulation buffers for optimal stability of Ven-A. Formulation in 20 mM acetate, 20 mM citrate, 20 mM histidine at pH 6 showed >87% of Ven-A recovery (FIG. 7). Similarly, 20 mM potassium phosphate pH 6 showed >91% main peak with <9% LMWS, indicating suitability as a formulation buffer (FIG. 7). A clear solution was observed in acetate and potassium phosphate buffers, and relative cloudiness was noted in Histidine and Citrate buffers. Further, citrate, phosphate and histidine formulations showed Tm values above 72° C., indicating high thermal stability (FIG. 8). Potential formulation buffers are citrate, histidine, potassium phosphate. However, Potassium Phosphate was selected for further formulation optimization. Overall, potassium phosphate>citrate>histidine listed most preferable to relatively less preferable. No thermal transition was observed when acetate buffer was used in the formulation (poor formulation buffer).

### Formulation of Ven-A Containing Stabilizing Agents

Stabilizing agents can stabilize the native conformation of proteins and suppress aggregation by interaction with the protein, water, air-water interfaces, and container surfaces. However, some stabilizing agents are also known to destabilize proteins. Hence, screening of stabilizing agents is critical for stable formulation development of proteins. Stabilizing agents were added to 20 mM potassium phosphate, pH 6.0 and formulations were subjected to stress at 40° C., 60% RH for 10 days.

### Formulations Containing Amino Acids

Amino acids can stabilize proteins by preferential exclusion, direct protein binding, providing buffer capacity, as well as anti-oxidant properties. For instance, arginine reduces viscosity and increases the solubility of proteins. Four amino acids were screened including lysine, L-arginine, L-glutamine, and glycine. The concentration of stabilizing agents was adjusted to 100 mM. Formulations containing Lysine and arginine showed 82% and 87% of the Ven-A recovery, respectively (FIG. 9). Similarly, both formulations that contained L-glutamine and glycine showed >90% main peak with no apparent HMWS (FIG. 9). Overall, formulations with lysine, L-arginine, and L-glutamine provided Ven-A recovery (FIG. 9). However, the two formulations with L-glutamine and glycine as stabilizing agents in 20 mM potassium phosphate pH 6.0 were found to be optimal. (FIG. 9). Ranking of stabilizing agents after evaluating % recovery, BMWS, and LMWS are L-glutamine >glycine >arginine >lysine.

### Formulations Containing Sugars and Polyol

The term “sugar” refers to monosaccharide, disaccharides, and polysaccharides. Examples of sugars include, but are not limited to, sucrose, glucose, dextrose, and others. Similarly, the term “polyol” refers to alcohol containing multiple hydroxy groups. Examples of polyols include, but are not limited to, mannitol, sorbitol, and others. Monosaccharides (sugars) exhibit repulsive interaction with the protein and are preferentially excluded from the protein surface, which favors the native conformational state. The repulsive interaction creates water shells around the protein, stabilizing the native state, while also decreasing propensity and rate of aggregation. Four formulations of Ven-A containing monosaccharides and polyols at 10% concentration were prepared. The stabilizing agents include: sucrose, trehalose, mannitol, and sorbitol were examined after incubation at 40° C., 60% RH for 10 days. A formulation containing sucrose showed 70% Ven-A recovery with 30% being LMWS (FIGS. 9-10). The high level of LMWS generated after stress indicates the destabilizing effect of sucrose. A formulation containing 10% trehalose showed 92% Ven-A recovery with a low level of LMWS, suggesting a stabilizing effect on Ven-A (FIGS. 9-10). Similarly, a formulation containing 10% mannitol, and 10% sorbitol both showed 92% Ven-A recovery, respectively (FIGS. 9-10). Therefore, trehalose, mannitol, and sorbitol as stabilizing agents demonstrated optimal stabilizing of Ven-A in the formulation. Ranking order: sorbitol trehalose, mannitol >sucrose. Overall, sucrose showed the lowest recovery compare to the other polyols and sugars.

### Formulations Containing Salts

Formulations containing salts may exhibit repulsive interaction with the protein and are preferentially excluded from the protein surface. Using preferential exclusion, salts regulate solubility and aggregation of proteins. The effect of salts on protein stability depends on the solution pH and type of ion used. Therefore, screening of salts to optimize formulation is necessary. In this study, common salts which include, sodium chloride and magnesium chloride were tested. Formulations of Ven-A containing 100 mM sodium chloride showed 94% main peak, indicating a stabilizing effect with minimal LMWS (FIG. 9). A similar result was observed with 10 mM magnesium chloride (FIG. 9). Therefore, both sodium chloride and magnesium chloride are good stabilizing agents and demonstrated excellent stability of Ven-A in the formulation (FIG. 9). Protein recovery measure using UV-VIS showed 84% and 93% formulation containing 100 mM sodium chloride and 10 mM magnesium chloride, respectively (FIG. 11). Taken all together, a formulation containing magnesium chloride or sodium chloride demonstrated optimal stability.

### Formulations Containing Carboxylic Acids

Formulation of Ven-A in 10 mM lactic acid and malic acid demonstrated 86% and 90% Ven-A recovery, respectively (FIG. 9). Formulation of Ven-A in 100 mM tartrate showed precipitation and was not further analyzed (poor stabilizing agent). A formulation containing 100 mM succinate showed 91% recovery, an excellent stabilizing effect (FIG. 9). The recovery was measured using UV-VIS for the formulation containing lactic, malic, and succinate showed 72%, 64%, and 90% Ven-A recovery, respectively (FIG. 11). Thus, based on UV-VIS and SEC data, succinate demonstrated the highest stabilizing effect on Ven-A formulation. Lactic and malic acid showed HMWS, while succinate showed no apparent HMWS. Ranking order: succinate >lactic and malic acids. Tartrate is a poor stabilizing agent and generally not preferred.

### Formulations Containing Surfactants

Non-ionic surfactants were used to stabilize the proteins by suppressing aggregation and assisting in protein folding (i.e., serving as chaperons). Surfactants were also used to protect the proteins from mechanical stress (e.g., shaking) induced aggregation and stabilize protein during freezing, and lyophilization processes. Ven-A formulations containing 0.2% (w/v) polysorbate 80, polysorbate 20, and pluronic F-68 in 20 mM potassium phosphate pH 6.0 were examined. Polysorbate 20 showed the highest stability with 86% Ven-A recovery (FIG. 9). Similarly, polysorbate 20 and pluronic F-68 showed, 77.4% and 49% Ven-A recovery on SEC-HPLC (FIG. 9). The Ven-A recoveries measured using UV-VIS for polysorbate 80, polysorbate 20, and pluronic F-68 (poor stabilizing agent), were 97%, 62%, and 27%, respectively (FIG. 11). A formulation containing polysorbate 80 showed excellent recovery and stability. Preferred formulation includes polysorbate 80>polysorbate 20>pluronic F68. Precipitation was observed in a formulation containing pluronic F-68. Hence pluronic F-68 is a poor stabilizing agent.

### Formulations Containing a Combination of Stabilizing Agents

L-glutamine, sorbitol, sodium chloride, and succinate were selected for further formulation optimization (FIG. 12). When stabilizing agents are combined in one formulation, the effect on protein stability could be positive or negative. Hence, designing and screening of different formulation combinations are necessary to confirm protein stability. Surfactant, polysorbate 80 was selected for further formulation optimization. At high concentration, surfactant destabilizes protein, while low concentrations may not sufficiently increase protein stability. Therefore, it is essential to determine the target concentration of surfactant that stabilizes proteins. From the previous study (i.e., screening), no significant chemical degradation was observed in the presence of the selected stabilizing agents; thus, formulation optimization was focused on physical stability. Formulations were subjected to agitation and freeze-thaw as stress conditions to identify the combination of stabilizing agents that provide optimal recovery and stability. The formulations were designed using full factorial experimental design (DOE, JMP 14.0.0). Sixteen formulations were examined using a combination of four selected stabilizing agents. Formulation with no stabilizing agent (F9) was examined as a control. The symbols (−) and (+) shows absence and presence of stabilizing agents in the formulation, respectively (Table 1). Formulations were prepared in the order generated from the full factorial DOE.

TABLE 1
The pattern of stabilizing agents (F1-F17) used for Ven-A formulations
in freeze-thaw and agitation stress conditions.

| | | Formulation Composition | | | | |
|---|---|---|---|---|---|---|
| | | | | | | Polysorbate 80, |
| Formulation Pattern | | L-Glutamine | Sorbitol | Sodium Chloride | Succinate | 0.05% |
| F1 | +−−+− | 100 | 0 | 0 | 100 | 0 |
| F2 | ++−−− | 100 | 10 | 0 | 0 | 0 |
| F3 | ++++− | 100 | 10 | 100 | 100 | 0 |
| F4 | −−+−− | 0 | 0 | 100 | 0 | 0 |
| F5 | −+++− | 0 | 10 | 100 | 100 | 0 |
| F6 | −+−+− | 0 | 10 | 0 | 100 | 0 |
| F7 | −−−+− | 0 | 0 | 0 | 100 | 0 |
| F8 | ++−+− | 100 | 10 | 0 | 100 | 0 |
| F9 | −−−−− | 0 | 0 | 0 | 0 | 0 |
| F10 | −++−− | 0 | 10 | 100 | 0 | 0 |
| F11 | −−++− | 0 | 0 | 100 | 100 | 0 |
| F12 | +−−−− | 100 | 0 | 0 | 0 | 0 |
| F13 | +−+−− | 100 | 0 | 100 | 0 | 0 |
| F14 | +−++− | 100 | 0 | 100 | 100 | 0 |
| F15 | −+−−− | 0 | 10 | 0 | 0 | 0 |
| F16 | +++−− | 100 | 10 | 100 | 0 | 0 |
| F17 | −−−−+ | 0 | 0 | 0 | 0 | 100 |

Formulation F1 showed 54% and 31% main peak Ven-A recovery after freeze-thaw and agitation, respectively, measured using SEC (FIGS. 14-15). Formulation F5, F6, F8, F10, F11, and F16 showed >80% Ven-A recovery, demonstrating higher stability during agitation (FIG. 15). In addition, these formulations showed >85% recovery based on UV-VIS characterization (FIGS. 14-15). Similarly, formulations F1, F2, F3, F5, F6, F8, F10, F11, F11, F13, and F16 demonstrated better than 80% Ven-A recovery based on UV-VIS (FIG. 16) demonstrating suitable formulation conditions for manufacturing and processing. After five freeze-thaw cycles, F5 and F10 showed Ven-A recovery better than 83% (FIGS. 16-17) indicating a stable formulation condition. Formulation F2, F3, F8, and F16 showed Ven-A recovery better than 65% (FIGS. 16-17) indicating a relatively stable formulation condition. Also, F3, F5, and F10 showed Ven-A recovery better than 81% by UV-VIS method (FIGS. 16-17) and were identified as desirable formulation conditions. Formulation without stabilizing agent 20 mM potassium phosphate pH 6.0 (F9) showed the lowest Ven-A recovery after agitation and freeze-thaw (FIGS. 14-17). Overall, most of the stabilizing agent combinations showed some stabilizing effect on Ven-A formulations (FIGS. 14-17).

Polysorbate 80 was formulated at a concentration of 0.025%, 0.05%, 0.1%, and 0.2% (w/v). A formulation containing 0.05% (w/v) polysorbate 80 showed >97% Ven-A recovery after freeze-thaw and 87% Ven-A recovery after agitation measured using UV-VIS (FIG. 18). Similarly, 0.05% (w/v) Polysorbate80 in 20 mM potassium phosphate showed 95% Ven-A recovery after freeze-thaw and 65% Ven-A recovery after agitation measured using SEC-HPLC (FIG. 19). In general, all concentrations of polysorbate-80 showed a stabilizing effect. Optimal recovery and stability of Ven-A were achieved at a concentration of 0.05% (w/v) polysorbate 80 in the formulation (FIGS. 18-19). Overall, polysorbate 80 showed stabilization at a concentration 0.025%-0.2% (w/v) and the ranking order is 0.05%>0.025%, 0.1%>0.2% (w/v).

Four formulations were selected that showed excellent recovery after being subjected to agitation and freeze-thaw stress conditions. The combination of stabilizing agents showed a synergistic stabilizing effect when compared to the formulation with no stabilizing agents (FIG. 20 and Table 2).

TABLE 2
Composition of the selected formulations for Ven-A.
Formulation nomenclature (F#) as in Table 1.

| Selected | | | Sodium | | Polysorbate 80, |
|---|---|---|---|---|---|
| formulations | L-Glutamine | Sorbitol | Chloride | Succinate | 0.05% |
| #1 (F3) | 100 | 10 | 100 | 100 | 0 |
| #2 (F5) | 0 | 10 | 100 | 100 | 0 |
| #3 (F10) | 0 | 10 | 100 | 0 | 0 |
| #4 (F17) | 0 | 0 | 0 | 0 | 100 |

The potency of Ven-A in the selected formulations was examined using a L929 cell-based bioassay with murine TNF-α (mTNF-α) and human TNF-α (hTNF-α). Overall, Ven-A showed high potency in all selected formulations (FIGS. 21-22). Ven-A was formulated at high concentrations (5 to 20 mg/ml) in the selected formulations. The data showed high recovery (>90%), indicating stable formulation conditions (FIG. 20).

### Proteolytic Degradation of Immunoglobulin A in SGF

To prepare simulated gastric fluid (SGF), 0.2 g of sodium chloride (NaCl) was dissolved in about 0.9 L of purified water. Next, the buffer was adjusted to pH 1.6 with 1N hydrochloric acid. To complete the preparation (see table 3 below), 0.12 g of fasted state simulated gastric fluid powder was dissolved in 1 liter of the buffer adjusted to pH 1.6 previously (producing FaSSGF at 2× concentration).

IgA formulations were prepared at a concentration of 4 mg/ml in HCl buffer pH 1.6 and mixed with 2× FaSSGF to make a final concentration of 2 mg/ml. Similarly, a control was prepared comprised of IgA at a concentration of 2 mg/mL in SGF without pepsin. Samples were incubated at 37° C. for 15 min and analyzed using denatured size exclusion chromatography (SEC).

TABE 3
The composition of the Fasted State Simulated Gastric Fluid
(FaSSGF or SGF).

| Composition | Concentration, mM |
|---|---|
| Sodium taurocholate | 0.08 |
| Lecithin | 0.02 |
| Sodium chloride | 34.2 |
| Hydrochloric acid | 28.4 |
| Pepsin | 0.1 mg/mL |
| pH | 1.6 |

### Proteolytic Degradation of Immunoglobulin A in SIF

In approximately 0.9 L of purified water, 0.42 g of sodium hydroxide pellets (NaOH), 3.95 g of monobasic sodium phosphate monohydrate (NaH₂PO₄.H₂O), and 6.19 g of sodium chloride (NaCl) were dissolved. Next, the buffer was adjusted to pH 6.5 with 1 N sodium hydroxide or 1N hydrochloric acid. To prepare the fasted state SIF (FaSSIF-V1), 4.48 g of the powder (i.e., 2× FaSSIF-V1) was dissolved in 1 liter of phosphate buffer pH 6.5. Pancreatin was added to the fluid to make a final concentration of 20 mg/ml (2×).

Except where noted, IgA formulations at a concentration of 4 mg/ml were prepared in the appropriate buffer (e.g., 20 mM potassium phosphate buffer) pH 6.0, 0.05% polysorbate-80 and mixed with the simulated fluid containing pancreatin (2×) to make a final concentration of 2 mg/ml. The final concentration of the pancreatin in the digestion was 10 mg/ml.

TABLE 4
The composition of the Fasted State Simulated Intestinal Fluid
(FaSSIF-V1 or SIF)

| Composition | Concentration, mM |
|---|---|
| Sodium taurocholate | 3 |
| Lecithin | 0.75 |
| Sodium chloride | 105.9 |
| Monobasic sodium phosphate | 28.4 |
| Sodium hydroxide | 8.7 |
| Pancreatin | 10 mg/mL |
| pH | 6.5 |

Similarly, control IgA samples were prepared at a concentration of 4 mg/mL in 20 mM potassium phosphate buffer pH 6.0, 0.05% polysorbate-80, and mixed with SIF (2×) without pancreatin to make a final concentration 2 mg/ml. Similarly, a control IgA at a concentration of 2 mg/ml in SIF without pancreatin was prepared. Samples were incubated at 37° C. for 30 min and analyzed using denatured size exclusion chromatography (SEC).

### Proteolytic Degradation of Immunoglobulin A in a SIF Containing Stabilizing Agents

IgA formulations at a concentration of 2 mg/ml were digested in SIF containing stabilizing agents for 1 hour. Also, samples were digested in SIF without stabilizing agents as a control. Peak area of the SEC chromatogram of intact IgA was monitored during digestion for all stabilizing agents.

### Proteolytic Degradation of Immunoglobulin A in SGF Containing Stabilizing Agents

IgA formulations at a concentration of 2 mg/mL were digested in SGF for 15 min containing stabilizing agents. Also, samples without stabilizing agents were used as a control and measured using denatured size exclusion chromatography. Digestion of the intact protein was monitored using denatured SEC.

### Protein L Affinity Chromatography

Formulations containing IgA and host expression system extract with and without digestion were purified using protein L chromatography. HPLC analysis was carried out using Shimadzu HPLC instrument, column with 1 ml of Toyopearl AF-rProtein L-650F resin was packed in-house, a flow rate of 1 ml/min and UV detection at 280 nm were used. Briefly, the column was equilibrated with 10 column volumes (CV) of the 10 mM sodium phosphate pH 7.0. Samples were adjusted to pH 7 before loading, and 200 μl was injected into the column. A step gradient was used with 100% 10 mM sodium phosphate pH 7.0 from 0-5 min, 100% 10 mM sodium phosphate, 0.5M NaCl, pH 7.0 from 5-10 min. To remove the high salt, the mobile phase was switched to 100% loading buffer from 10-15 min. Finally, IgA was eluted using 20 mM sodium phosphate pH 2.0 from 15-22 min, and fractions of 1 ml were collected and concentrated for further analysis using SEC. The column was equilibrated with 10 CV loading buffer for subsequent runs.

### Formulations of Immunoglobulin A Containing Natural Proteins as Stabilizing Agents

Base formulations containing purified IgA, 0.05% polysorbate-80, and 20 mM potassium phosphate buffer pH 6.0 were combined with various natural proteins as stabilizing agents: albumin, α-lactalbumin, casein, whey, lactoferrin, lysozyme, and tested in SIF and SGF. Also, tryptone was used for stabilization of IgA in both fluids. The concentrations used in the formulations of lactoferrin, α-lactalbumin, lysozyme, and tryptone were 50 mg/ml. Similarly, the concentration of albumin, whey, and casein in the formulations were 18 mg/ml, 25 mg/ml, and 10 mg/ml, respectively. The IgA concentration in all the formulations tested for stability was kept at 2 mg/ml, and the digestion time was 15 min in SGF and 30 min in SIF. The stability of IgA was monitored using SEC.

TABLE 5
IgA Formulations-Natural Proteins. Formulation nomenclature
(F#) continues from Tables 1 and 2.

| | | SIF (30 min) | SGF (15 mm) |
|---|---|---|---|
| | | % Intact Protein | % Intact Protein |
| Formulation | Composition | by SEC | by SEC |
| Ref 1 | IgA Control | (no pancreatin) 100 | (no pepsin) 100 |
| Ref 2 | IgA without | 32 | 9 |
| | stabilizing agent | | |
| | proteins | | |
| F18 | IgA + Albumin | 36 | 71 |
| F19 | IgA + Casein | 60 | 74 |
| F20 | IgA + α-lactalbumin | 87 | 78 |
| F21 | IgA + Lactoferrin | 80 | 59 |
| F22 | IgA + Lysozyme | 80 | 68 |
| F23 | IgA + Tryptone | 72 | 70 |
| F24 | IgA + Whey | 80 | 71 |

### Formulations of Immunoglobulin A Containing Surfactants

Base formulations containing purified IgA, 0.05% polysorbate-80, and 20 mM potassium phosphate buffer pH 6.0 were combined with various surfactants: caprylyl sulfobetaine, lauryl sulfobetaine, myristyl sulfobetaine, and stearyl sulfobetaine. A stock solution with a concentration of 6.25% (w/v) was prepared for each surfactant. Formulations were prepared to contain 2 mg/ml of IgA and 2% (w/v) of the surfactants. Samples were digested for 15 minutes in SGF and 30 minutes in SIF. The proteolytic degradation of IgA was monitored using size exclusion chromatography.

TABLE 6
IgA Formulations-Surfactants. Formulation nomenclature (F#)
continues from Table 5.

| | | SGF (15 min) | SIF (30 min) |
|---|---|---|---|
| | | % Intact | % Intact |
| | | Protein | Protein |
| Formulation | Composition | by SEC | by SEC |
| Ref 1 | IgA, Control | (no pepsin) | (no pancreatin) |
| | | 100 | 100 |
| Ref 2 | IgA without surfactant | 9 | 32 |
| F25 | IgA + Caprylyl Sulfobetaine | 42 | 68 |
| F26 | IgA + Lauryl Sulfobetaine | 82 | 51 |
| F27 | IgA + Myristyl Sulfobetaine | 89 | 77 |
| F28 | IgA + Stearyl Sulfobetaine | 51 | 78 |

### Formulations of Immunoglobulin A Containing Buffers

Formulations of purified IgA with 0.05% (w/v) polysorbate-80 (except as noted) were prepared using different buffer systems. IgA was prepared at 4 mg/ml, in 20 mM phosphate buffer pH 6.0, 250 mM potassium phosphate buffer pH 6.0, 250 mM histidine buffer pH 6.0, 1000 mM histidine buffer pH 6.0, 1000 mM histidine buffer pH 5.0, and 1000 mM histidine buffer pH 4.0. The stability of the formulations was tested in SGF.

TABLE 7
IgA Formulations-Buffers. Formulation nomenclature (F#)
continues from Table 6.

| Formulations | Composition | % Intact Protein |
|---|---|---|
| Ref 3 | IgA Control without pepsin | 100 |
| Ref 4 | IgA without stabilizing buffer | 5 |
| F29 | IgA + 10 mM Bicarbonate pH 9.0 | 37 |
| | (without polysorbate-80) | |
| F30 | IgA + 20 mM Potassium Phosphate pH 6.0 | 69 |
| F31 | IgA + 250 mM Potassium Phosphate pH 6.0 | 91 |
| F32 | IgA + 250 mM Histidine pH 6.0 | 93 |
| F33 | IgA + 1000 mM Histidine pH 6.0 | 103 |
| F34 | IgA + 1000 mM Histidine pH 5.0 | 69 |
| F35 | IgA + 1000 mM Histidine pH 4.0 | 17 |

Formulations of Immunoglobulin A with Host Expression System Extract

Unpurified IgA extract from the host recombinant expression system (rice) was dissolved in 20 mM potassium phosphate buffer pH 6.0, and 0.05% (w/v) polysorbate-80 and tested in SGF.

TABLE 8
IgA Formulations-Host expression protein extract. Formulation
nomenclature (F#) continues from Table 7.

| | | % Intact |
|---|---|---|
| Formulations | Composition | Protein |
| F36 | Control, IgA extract + SGF (pH 1.6) | 39 |
| F37 | IgA + 20 mM Potassium Phosphate pH 6.0 + 0.05% | 88 |
| | (w/v) Polysorbate 80 + grain extract + SIF | |
| F38 | IgA extract + 20 mM Potassium Phosphate | 74 |
| | pH 6.0 + 0.05% (w/v) Polysorbate-80 | |
| F39 | IgA extract + 500 mM Histidine pH 6.0 + 0.05% | 81 |
| | (w/v) Polysorbate-80 + SGF | |
| F40 | IgA extract + 500 mM Histidine pH 6.0 + 0.05% | 73 |
| | (w/v) Polysorbate-80 + SIF | |

Stabilization of a Preferred sIgA (Ven-A) in an Animal Model

The efficacy of orally delivered Ven-A is analyzed in a Dextran sodium sulfate (DSS)-induced animal model of inflammatory bowel disease (IBD). Stabilizing formulation F41 can be used: 250 mM Histidine pH 6.0, 0.05% w/v Polysorbate 80, 50 mM Potassium Chloride. Ten groups of C57BL/6 female, 6-8 week-old mice are utilized in this study, with ten mice per group. The groups are classified as follows:
1. C57BL/6 mice no DSS
2. C57BL/6 mice, 1.25% DSS ad libitum
3. C57BL/6 mice, 1.25% DSS ad libitum treated with Formulation such as F41, no Ven-A, 200 μL oral gavage daily
4. C57BL/6 mice, 1.25% DSS ad libitum treated with 50 mg/kg Cyclosporin A in 8% Cremophor EL, 5% EtOH in water, 200 μL oral gavage daily
5. C57BL/6 mice, 1.25 DSS ad libitum treated with 5 mg/kg Humira (adalimumab), IP every 3^{rd} day
6. C57BL/6 mice, 1.25% DSS ad libitum treated with 1000 μg recombinant albumin in 50 mM Tris, 150 mM NaCl, 0.1% Tween-20; pH 7.5, 200 μL oral gavage daily
7. C57BL/6 mice, 1.25% DSS ad libitum treated with 1000 μg recombinant formulated Ven-A, in, for example, F41, 200 μL oral gavage daily
8. C57BL/6 mice, 1.25% DSS ad libitum treated with 300 μg recombinant formulated Ven-A, in, for example, F41; pH 7.5, 200 μL oral gavage daily
9. C57BL/6 mice, 1.25% DSS ad libitum treated with 100 μg recombinant formulated Ven-A, in, for example, F41, 200 μL oral gavage daily
10. C57BL/6 mice, 1.25% DSS ad libitum treated with 30 μg recombinant formulated Ven-A, in, for example, F41, 200 μL oral gavage daily
   At day 0, mice are treated with DSS (1.25% w/v) in drinking water for the following 6 days. Fresh DSS solution is replaced at day 3, midway through the study. In addition, starting at day 0, DSS-treated groups will receive daily gavage of Cyclosporin, sham buffers or controls, or Ven-A, at the same time each day of administration. An additional control, adalimumab, is delivered intraperitoneally every 3rd day. At day 6, DSS solution is replaced with drinking water. Mice are monitored for weight loss and stool blood content to day 10. Mice are subsequently sacrificed, immediately prior to which time blood is collected by cardiac puncture and allowed to clot. Blood serum is reserved for serum chemistry analysis and for determination of systemic Ven-A levels. In addition, feces and luminal contents are collected for Ven-A measurement. Moreover, spleen, mesenteric lymph node and colonic tissue are excised for additional inflammatory profiling by leukocyte flow cytometric analysis.

Postmortem histology is carried out and includes H&E staining, Alcan blue staining, and immunohistochemical evaluation of lymphocyte infiltration of intestinal and colon tissues. The latter includes specific staining for CD3+T-lymphocytes, CD8+T-lymphocytes, CD68+ monocytes/macrophages, and neutrophil granulocytes (Myeloperoxidase marker). Histological parameters are measured including infiltration, crypt destruction, and goblet cell loss. In addition, a scoring system is applied to grade multiple histological parameters, as follows: severity of inflammation (0-3: none, slight, moderate, severe), extent of injury (0-3: none, mucosal, mucosal and submucosal, transmural), and crypt damage (0-4: none, basal ⅓ damaged, basal ⅔ damaged, only surface epithelium intact, entire crypt and epithelium lost). The score of each parameter is multiplied by a factor reflecting the percentage of tissue involvement (x1: 0-25%, x2: 26-50%, x3: 51-75%, x4: 76-100%). The maximum possible score is 40. Additional non-histological parameters are recorded, including body weight loss, colon length, reduction of spleen size, and extent of intestinal bleeding.

Pharmacokinetic profiling is performed on samples collected from the Caecum and small intestine. After opening and collection of the whole caecum content, contents are weighed, resuspended in collection buffer (PBS+0.5M EDTA, 0.1 mg/mL Soybean Trypsin inhibitor, PMSF) and homogenized. Intestinal contents are collected by lavage with collection buffer, centrifuged and filtered to remove bacterial contaminants. Faecal samples are similarly weighed, resuspended in collection buffer, homogenized and centrifuged. All collected samples are stored at −20° C. for further analysis.

For flow cytometric analysis, cells are isolated from lymph nodes, spleen, and colonic lamina propria. Mesenteric lymph nodes are pressed against a 70-μm cell strainer for cell collection. For colonic cell collection, colons are opened along the mesentery and luminal content is rinsed off before cutting the tissue into 1-cm sections in PBS+15 mM HEPES/1 mM EDTA. The tissue is vortexed and passed through a 70-μm tissue strainer. Remaining tissue is digested using 1 mg/mL type VIII collagenase, refiltered through a 70-μm cell strainer and used for cell counting. Colonic CD4⁺ cells are isolated by positive selection for flow cytometry. Cells from all three compartments are characterized using flow-cytometry-certified fluorescent antibodies against CD4, Interferon-gamma, CD25 and FoxP3.

Stabilization of a Preferred sIgA (Ven-B) in an Animal Model Study

A preclinical animal model study was carried out following similar protocols to those described above. To test whether formulations such as those described have a beneficial effect in an in vivo context, Ven-B, a preferred form of sIgA, was formulated in a variant of F32, termed F41 (250 mM Histidine pH 6.0, 0.05% w/v Polysorbate 80, 50 mM Potassium Chloride). As described above, Ven-B comprises a set of recombinant human monoclonal sIgA molecules of allotypes IgA1 or IgA2 ml that specifically target the surface antigen of Enterotoxigenic Escherichia coli (ETEC), which mediates binding between the pathogenic ETEC bacterium and host intestinal epithelial cells, allowing the bacterium to colonize the gut. Binding of any of the Ven-B antibodies to ETEC cfaE is intended to block the attachment of ETEC bacteria to human gut epithelia and prevent ETEC-induced diarrhea and associated symptoms. A member of the Ven-B antibody set was formulated in F41 and in a control formulation of 1× Phosphate Buffered Saline (F42) and tested in a preclinical experimental animal model of ETEC infection. Ven-B formulated in F41 was orally administered at a dosage of 10 mg/kg bodyweight of each animal, and was highly clinically effective (100%) in this preclinical animal (mouse) model (FIG. 30), meaning that ETEC-induced diarrhea and associated symptoms was prevented in 100% of the treated mice. In comparison, Ven-B formulated in the control F42 formulation exhibited a substantially poorer clinical effectiveness score. The ETEC challenge was carried out simultaneously with administration of the test formulations.

TABLE 9
IgA Formulations-ETEC preclinical animal model study. Formulation
nomenclature (F#) continues from Table 8.

| | | % Clinical |
|---|---|---|
| Formulations | Composition | Effectiveness |
| F41 | 250 mM Histidine pH 6.0 + 0.05% (w/v) | 100 |
| | Polysorbate-80 + 50 mM Potassium Chloride | |
| F42 | 1x Phosphate Buffered Saline: 137 mM | 25 |
| | Sodium Chloride + 2.7 mM Potassium | |
| | Chloride +10 mM Di-Sodium Hydrogen | |
| | Phosphate + 1.8 Potassium Di-Hydrogen | |
| | Phosphate pH 7.4 | |

### Discussion

### Natural Proteins as Stabilizing Agents

Natural proteins were selected as stabilizing agents to stabilize IgA under intestinal and gastric conditions. Such stabilizing agents tested include albumin, casein, α-lactoalbumin, lactoferrin, lysozyme, whey as well as tryptone. Stabilizing agents were tested for their stabilizing effect under intestinal and gastric conditions. Formulations without stabilizing natural proteins were tested as controls. The degradation of IgA in SIF as well as SGF without natural protein stabilizing agents was rapid, exemplifying the harsh proteolytic conditions. However, formulations containing natural proteins showed significant stability after digestion in intestinal and gastric fluids for 30 min and 15 min, respectively. Differences in stability between the formulation comprising stabilizing agents were noted. The percentage of intact IgA recovered after digestion is as follows (FIG. 24): formulation containing α-lactalbumin >87%, lactoferrin, lysozyme, and whey >80%, while formulations containing tryptone and casein showed greater than 72% and 60%, respectively. Those formulations comprising natural proteins can also contain additional stabilizing agents such as surfactant and buffers to provide further stabilization. Overall, the ranking of the natural proteins in stabilizing IgA is in the order of α-lactalbumin >lactoferrin >lysozyme and whey >tryptone >casein.

The stability of formulations containing natural proteins was also tested under gastric condition. Formulations of IgA without stabilizing natural protein degraded rapidly within 15 min and showed 9% of intact IgA by size exclusion chromatography. This data showed the need to design formulation to stabilize IgA for effective treatment of diseases. Formulations containing albumin, α-lactalbumin, casein, lactoferrin, lysozyme, whey, and tryptone were tested and showed >68% of intact IgA measured by size exclusion chromatography (FIG. 25). A formulation containing lactoferrin showed >59% of intact IgA after digestion. Ranking order of the stabilizing agents in stabilization of IgA is as follows: α-lactalbumin >casein>whey, tryptone and albumin >lysozyme >lactoferrin (FIG. 25). Two or more natural proteins can be used in a single formulation. Also, these natural proteins can be used with other stabilizing agents such as surfactants, and buffers to deliver stable IgA for treatment in the intestine and colon. The concentration of natural proteins in the formulation can be greater than 1 mg/ml, preferably greater than 10 mg/ml, even more preferably greater than 36 mg/ml. High concentration of those natural proteins is known to be safe for humans, although high levels of casein showed precipitation in the formulation.

### Surfactant Stabilizers

Pharmaceutically acceptable surfactants were used to stabilize IgA in the gastric and intestinal fluids. Such surfactants include caprylyl sulfobetaine, lauryl sulfobetaine, myristyl sulfobetaine, and stearyl sulfobetaine. The concentration of surfactant used was greater than 2% (w/v). The percentage of surfactant in the formulation can be greater than 2% (w/v), preferably >0.05% (w/v), and more preferably >1% (w/v). After 15 min of digestion in gastric conditions, formulations with caprylyl sulfobetaine and stearyl sulfobetaine showed that the percent of intact (recovered) IgA was greater than 42% and 51%, respectively (FIG. 26). Formulations containing lauryl sulfobetaine and myristyl sulfobetaine showed 82% and 89% of intact IgA, respectively. The ranking order of the surfactants in stabilization of IgA is as follows: myristyl sulfobetaine >lauryl sulfobetaine >stearyl sulfobetaine >caprylyl sulfobetaine (FIG. 26).

Formulations containing surfactants were also tested for their stability in intestinal conditions. Significant stability was noted for formulations containing surfactant when compared with the same formulation without a stabilizing agent. Formulations containing stearyl sulfobetaine and myristyl sulfobetaine showed greater than 78% and 77% of intact IgA, respectively (FIG. 27). Similarly, formulations comprising caprylyl sulfobetaine and lauryl sulfobetaine showed 68% and 51% of intact IgA, respectively. The ranking order of the surfactants in stabilization of IgA is as follows: stearyl sulfobetaine >myristyl sulfobetaine >caprylyl sulfobetaine >lauryl sulfobetaine (FIG. 27). Two or more surfactants can be used together with the other stabilizing agents such as natural proteins and stabilizing buffers.

In general, precipitation was noted with sodium dodecyl sulfate. Also, surfactants at low concentration (<0.8%) demonstrated poor protection.

### Stabilizing Buffers

Several different buffers were used to stabilize IgA including 10 mM bicarbonate, 20 mM potassium phosphate pH 6.0, 250 mM potassium phosphate, and 250 mM histidine pH 6.0. A formulation containing 20 mM potassium phosphate pH 6.0 showed 62% of intact IgA, while 10 mM bicarbonate (without polysorbate-80) at pH 9, showed 37% intact IgA after 15 minutes of digestion in SGF (Table 7; FIG. 28). Formulations with 250 mM potassium phosphate pH 6.0 showed nearly 100% of intact IgA, as did formulations with 250 mM histidine, pH 6.0 and 1000 mM histidine, pH 6.0. A1000 mM histidine buffer was mixed 1:9 (v/v) with the SGF media. The concentration of prepared IgA was 20 mg/ml which was mixed with SGF to make a final concentration of 2 mg/ml. In comparison, 1000 mM histidine, pH 5.0, 1000 mM histidine, pH 4.0, showed about 69% and 17% of intact IgA, respectively, after 15 minutes of digestion in SGF (i.e., 1:9 (v/v)) (FIG. 28). These results showed higher stability of IgA in the developed formulations buffered at pH 6.0. Overall, the formulations are ranked as follows: 1000 mM histidine pH 6.0, 250 mM potassium phosphate pH 6.0, 250 mM histidine pH 6.0, >20 mM potassium phosphate pH 6.0, >10 mM bicarbonate buffer pH 9 (FIG. 28). Other comparable buffers are citrate, acetate, tris, glycine and arginine prepared at a concentration of greater than 5 mM, preferably greater than 50 mM, and more preferably greater than 500 mM.

### Protein Expression System Extract

Instead of using purified IgA from the rice expression system as in the foregoing formulations, unprocessed IgA extract from the host expression system can be used in oral stability formulations. In the examples, the extract included the aqueous protein extraction fluid from the rice expression system, and related soluble fractions, including IgA extracted from the expression system. It will be appreciated that this extract can also include starches/carbohydrates, fats, fibers, and other proteins depending upon the expression system used. The extract is preferably used in its raw/unprocessed state, although it can be filtered or concentrated. Extraction fluids include water, salts, and/or appropriate buffer systems.

Formulations containing the expression extract, digested in SIF, showed nearly 100% recovery of the intact IgA after 30 minutes of digestion in SIF (FIG. 29). This data showed stability of IgA when formulated in plant extract which is a potential therapeutic application. When further formulated with 20 mM potassium phosphate pH 6.0 pH 6.0, and 0.05% polysorbate-80, the formulation showed >81% of intact protein (FIG. 29). Without the buffer system, recovery was only 30% in SGF.

### Inhibitors for Stabilization

Various inhibitors as stabilizing agents were tested in the simulated intestinal and gastric fluids. The stabilizing agents tested include trypsin inhibitor, puromycin dihydrochloride, aprotinin, and N-acetyl cysteine. The concentration of the stabilizing agents used in the formulation was 0.1 mg/mL. Formulations containing inhibitors showed no detectable main peak indicating significant proteolytic degradation and unstable composition. Thus, inhibitors were not explored further. Compared to other class of stabilizing agents, inhibitors provide no protection to IgA in simulated intestinal and gastric conditions.

sIgA Stabilization by Formulation During Therapeutic Delivery

As noted, a preferred example of sIgA, Ven-B, was formulated in a formulation F41 (which contains Histidine pH 6.0, 0.05% polysorbate-80 and potassium chloride) for testing by oral administration in an experimental preclinical animal model of Enterotoxigenic Escherichia coli (ETEC) infection. While the same sIgA in a control solution, phosphate buffered saline, was ineffective, the stabilized formulation of Ven-B that was delivered orally exhibited high clinical effectiveness (FIG. 30). These data showed stability and preservation of function, likely including antigen binding, in a context that closely approximates therapeutic application of sIgA. In particular, the data are consistent with sIgA stability during oral delivery.

It will be appreciated that this and other formulations described are intended to similarly stabilize sIgA in other therapeutic applications. For example, Ven-A, as described above, is a recombinant human monoclonal sIgA molecule that specifically binds to human TNF-α. This preferred sIgA could be formulated in F41 or other described stabilizing formulations for oral administration in experimental animal models of diseases characterized by chronic inflammation of the digestive system, in particular the small and large intestine. Such disease include inflammatory bowel disease, colitis, and Crohn's disease. Stabilization of Ven-A during oral delivery would increase survival and resistance of the sIgA to proteolysis in the stomach and in the intestine. and increased delivery of active Ven-A to sites of inflammation and TNF-α deposition in the intestinal lining (lamina propria and intima). Increased survival and delivery of Ven-A to sites of action would confer the benefit of promoting more effective anti-inflammatory effect and therapeutic treatment with smaller sIgA doses and fewer side effects compared to intravenous or IV infused antibody, the typical standard of care. Development of an orally active antibody targeting pro-inflammatory cytokines has been elusive until now because of the quick degradation of target antibodies in the gastrointestinal tract.

1. A stabilized prophylactic and/or therapeutic formulation comprising a therapeutically-effective amount of immunoglobulin A (IgA) dispersed in a pH buffering agent at a pH of from about 5 to about 8, wherein said formulation further comprises an optional non-ionic surfactant and one or more optional stabilizing agents, wherein said formulation exhibits physical and chemical stability after mechanical agitation and/or a freeze/thaw cycle.

2. The stabilized prophylactic and/or therapeutic formulation of claim 1, wherein the formulation remains stable during manufacturing, purification, and storage, and has at least 50% recovery of stable immunoglobulin.

3. The stabilized prophylactic and/or therapeutic formulation of claim 2, wherein the formulation has at least 50% recovery of stable immunoglobulin under storage conditions at about 25° C. and 60% RH for up to about 6 months.

4.-5. (canceled)

6. The stabilized prophylactic and/or therapeutic formulation of claim 1, wherein said formulation comprises up to about 200 mg/ml IgA.

7. The stabilized prophylactic and/or therapeutic formulation of claim 1, wherein said IgA is monomeric IgA, dimeric IgA, sIgA, glycosylated or non-glycosylated forms of IgA, chemical variants, recombinant forms, minor mutants thereof, or combinations thereof.

8. The stabilized prophylactic and/or therapeutic formulation of claim 7, wherein said IgA is sIgA.

9. The stabilized prophylactic and/or therapeutic formulation of claim 8, wherein said sIgA is a recombinant sIgA.

10. The stabilized prophylactic and/or therapeutic formulation of claim 9, wherein said recombinant sIgA is expressed in a plant system.

11. The stabilized prophylactic and/or therapeutic formulation of claim 10, said formulation comprising a combination of recombinant sIgA and monomeric IgA expressed in said plant system.

12. The stabilized prophylactic and/or therapeutic formulation of claim 10, wherein said plant system is a monocot.

13. The stabilized prophylactic and/or therapeutic formulation of claim 10, wherein plant system is selected from the group consisting of wheat (Triticum sps.), rice (Oryza sps.), barley (Hordeum sps.), oats (Avena sps.), rye (Secale sps.), corn (maize) (Zea sps.), and millet (Pennisettum sps.), triticale, and sorghum.

14.-15. (canceled)

16. The stabilized prophylactic and/or therapeutic formulation of claim 1, wherein said pH buffering agents are selected from the group consisting of potassium phosphate, citrate, histidine, acetate, bicarbonate, and combinations thereof.

17. (canceled)

18. The stabilized prophylactic and/or therapeutic formulation of claim 1, wherein said non-ionic surfactants are present and selected from the group consisting of polysorbate 80 (polyoxyethylene (20) sorbitan monooleate), polysorbate 20 (polyoxyethylene (20) sorbitan monolaurate), and combinations thereof.

19.-20. (canceled)

21. The stabilized prophylactic and/or therapeutic formulation of claim 1, said formulation further comprising at least one of said stabilizing agents selected from the group consisting of: amino acids, sugars/polyols, chloride salts, carboxylic acids, detergents, natural proteins, protein expression extracts, and mixtures thereof.

22.-25. (canceled)

26. The stabilized prophylactic and/or therapeutic formulation of claim 21, wherein said chloride salts for use in the formulations are selected from the group consisting of sodium chloride, magnesium chloride, potassium chloride, calcium chloride, and combinations thereof.

27.-30. (canceled)

31. The stabilized prophylactic and/or therapeutic formulation of claim 21, wherein said detergents for use in the formulations are zwitterionic detergents, selected from the group consisting of caprylyl sulfobetaine, lauryl sulfobetaine, myristyl sulfobetaine, stearyl sulfobetaine, and combinations thereof.

32. (canceled)

33. The stabilized prophylactic and/or therapeutic formulation of claim 21, wherein said natural proteins for use in the formulations are selected from the group consisting of albumin, α-lactalbumin, casein, whey, lactoferrin, lysozyme, tryptone, and combinations thereof.

34. (canceled)

35. The stabilized prophylactic and/or therapeutic formulation of claim 21, wherein said immunoglobulin is a recombinant IgA expressed in a host system, wherein said protein expression extract comprises said IgA and protein extraction fluid from extracting said IgA from said host system.

36. The stabilized prophylactic and/or therapeutic formulation of claim 35, wherein said protein expression extract is unpurified.

37.-38. (canceled)

40. The stabilized prophylactic and/or therapeutic formulation of claim 1, wherein said formulation is substantially free of sucrose, tartrate, and/or pluronic F68.

41. The stabilized prophylactic and/or therapeutic formulation of claim 1, comprising IgA, potassium phosphate buffering agent, polysorbate 80, and either α-lactalbumin or Myristyl Sulfobetaine.

42. (canceled)

43. The stabilized prophylactic and/or therapeutic formulation of claim 1, comprising recombinant IgA in protein expression extract, potassium phosphate or histidine buffering agent, and polysorbate 80.

44. The stabilized prophylactic and/or therapeutic formulation of claim 1, comprising IgA, histidine buffering agent pH 6.0+/−0.2, and polysorbate-80, optionally further comprising monovalent chloride salt.

45.-47. (canceled)

48. A prophylactic or therapeutic method comprising administering a therapeutically effective amount of a stabilized prophylactic and/or therapeutic formulation according to claim 1 to a subject in need thereof.

49.-59. (canceled)

60. The method of claim 48, wherein said formulation is administered as a food supplement, such as in infant formula or a probiotic supplement.


SEQUENCE LISTING (9 sequences)

SEQ ID NO: 1 - protein, 87 aa, Homo sapiens
  MISC_FEATURE (1)..(87): kappa variable region
DIQMTQSPSTLSASVGDRVTITCRASQSINTWLAWYQQKPRFIGSGSGTEFTLTISSLQPDDFATYYCQQYNSDSKMFGQGTKVEVK

SEQ ID NO: 2 - protein, 107 aa, Homo sapiens [sequence omitted]

SEQ ID NO: 3 - protein, 115 aa, Homo sapiens
  MISC_FEATURE (1)..(115): alpha variable region
EVQLVETGGGLIQPGGSLRLSCAASGFTVSNHSMSWVRQAPGKALEWVSAIYRGGTTYYADSVKGRFTISRDDSRNTVYLQMNSLRAEDTAVYYCARDLAAARLFGKGTTVTVSS

SEQ ID NO: 4 - protein, 353 aa, Homo sapiens
  MISC_FEATURE (1)..(353): alpha1 constant region
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 5 - protein, 340 aa, Homo sapiens
  MISC_FEATURE (1)..(340): alpha2m1 constant region
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 6 - protein, 340 aa, Homo sapiens
  MISC_FEATURE (1)..(340): alpha2m2 constant region
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 7 - protein, 340 aa, Homo sapiens
  MISC_FEATURE (1)..(340): alpha2n constant region
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 8 - protein, 137 aa, Homo sapiens
  MISC_FEATURE (1)..(137): joining chain
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 9 - protein, 585 aa, Homo sapiens
  MISC_FEATURE (1)..(585): secretory component
KSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPR